(19) United States
(12) Patent Application Publication
(10) Pub. No.: US 2022/0389392 A1
(43) Pub. Date: Dec. 8, 2022

(54) CRISPR CAS SYSTEMS AND LYSOGENY MODULES

(71) Applicant: Locus Biosciences, Inc., Morrisville, NC (US)

(72) Inventors: David G. OUSTEROUT, Morrisville, NC (US); Paul M. GAROFOLO, Morrisville, NC (US); Kurt SELLE, Morrisville, NC (US); Hannah Hewitt TUSON, Morrisville, NC (US)

(21) Appl. No.: 17/774,309

(22) PCT Filed: Nov. 5, 2020
(86) PCT No.: PCT/US2020/059213

§ 371 Date: May 4, 2022

Related U.S. Application Data
(60) Provisional application No. 62/931,793, filed on Nov. 6, 2019.

(51) Int. Cl. C12N 7/00; C12N 15/11; C12N 9/22; A61P 31/04; A61K 35/76

(52) U.S. Cl. CPC C12N 7/00; C12N 15/11; C12N 9/22; A61P 31/04; A61K 35/76; C12N 2795/10343; C12N 2310/20; C12N 2800/80; C12N 2795/10371

(57) ABSTRACT

Disclosed herein are compositions and methods for modifying a bacterial population. In some embodiments, described herein is a bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic.

### CROSS-REFERENCE

This application claims the benefit of U.S. Patent Application No. 62/931,793, filed Nov. 6, 2019, which is hereby incorporated by reference in its entirety.

### SUMMARY

In some embodiments, described herein is a bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. In some embodiments, the bacteriophage is derived from a temperate bacteriophage. In some embodiments, the bacteriophage is rendered lytic by removal, replacement, or inactivation of a lysogenic gene. In some embodiments, the bacteriophage is rendered lytic by removal of a 1247 cI repressor region. In some embodiments, the bacteriophage is rendered lytic by the removal of a 1249 cI repressor region. In some embodiments, the bacteriophage is rendered lytic by the removal of a 1224 cI repressor region. In some embodiments, the bacteriophage is rendered lytic by the removal of a regulatory element of a lysogeny gene. In some embodiments, the bacteriophage is rendered lytic by the removal, alteration or replacement of a promoter of a lysogeny gene. In some embodiments, the bacteriophage is rendered lytic by the removal of a functional element of a lysogeny gene. In some embodiments, the bacteriophage is rendered lytic via a second CRISPR array comprising a second spacer directed to a lysogenic gene. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential bacterial gene that is needed for survival of the target bacterium. In some embodiments, the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. In some embodiments, the target nucleotide sequence is in a non-essential bacterial gene or genomic locus. In some embodiments, the first nucleic acid sequence is a first CRISPR array further comprising at least one repeat sequence. In some embodiments, the at least one repeat sequence is operably linked to the first spacer sequence at either its 5′ end or its 3′ end. In some embodiments, the first nucleic acid is inserted into a non-essential bacteriophage gene or other genomic locus. In some embodiments, the non-essential gene is gp49, gp75, hoc, gp0.7, gp4.3, gp4.5, gp4.7, gp0.6, gp0.65, gp0.7, gp4.3, or gp4.5. In some embodiments, the target bacterium is C. difficile. In some embodiments, the bacteriophage is ϕCD146 or ϕCD24-2. In some embodiments, the target bacterium is killed by the lytic activity of the bacteriophage, by the activity of a CRISPR-Cas system using the first spacer sequence or the crRNA transcribed therefrom, or both. In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system, a Type II CRISPR-Cas system, or a Type III CRISPR-Cas system. In some embodiments, the CRISPR-Cas system is a Type I CRISPR-Cas system.

In some embodiments, disclosed herein is a temperate bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the 1247 cI repressor region. In some embodiments, the temperate bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential bacterial gene that is needed for survival of the target bacterium. In some embodiments, the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. In some embodiments, the target nucleotide sequence is in a non-essential bacterial gene or genomic locus. In some embodiments, the first nucleic acid sequence is a first CRISPR array further comprising at least one repeat sequence. In some embodiments, the at least one repeat sequence is operably linked to the first spacer sequence at either its 5′ end or its 3′ end. In some embodiments, the first nucleic acid is inserted into a non-essential bacteriophage gene or other genomic locus. In some embodiments, the non-essential bacteriophage gene is gp49, gp75, hoc, gp0.7, gp4.3, gp4.5, gp4.7, gp0.6, gp0.65, gp0.7, gp4.3, or gp4.5. In some embodiments, the target bacterium is C. difficile. In some embodiments, the temperate bacteriophage is ϕCD146 or ϕCD24-2. In some embodiments, the target bacterium is killed by the lytic activity of the temperate bacteriophage, by the activity of a CRISPR-Cas system using the first spacer sequence or the crRNA transcribed therefrom, or both. In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system, a Type II CRISPR-Cas system, or a Type III CRISPR-Cas system. In some embodiments, the CRISPR-Cas system is a Type I CRISPR-Cas system. In some embodiments, disclosed herein is a pharmaceutical composition comprising: (a) a bacteriophage as disclosed herein, or a temperate bacteriophage as disclosed herein; and (b) a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, or any combination thereof.

In some embodiments, disclosed herein is a method for killing a target bacterium, the method comprising introducing into the target bacterium a temperate bacteriophage comprising a first nucleic acid sequence encoding a first spacer sequence or a crRNA transcribed therefrom, wherein the first spacer sequence is complementary to a target nucleotide sequence from a target gene in the target bacterium, provided that the bacteriophage is rendered lytic by a 1247 cI repressor region knockout, thereby killing the target bacterium. In some embodiments, the temperate bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential bacterial gene that is needed for survival of the target bacterium. In some embodiments, the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. In some embodiments, the target nucleotide sequence is in a non-essential bacterial gene or genomic locus. In some embodiments, the first nucleic acid sequence is a first CRISPR array further comprising at least one repeat sequence. In some embodiments, the at least one repeat sequence is operably linked to the first spacer sequence at either its 5′ end or its 3′ end. In some embodiments, the first nucleic acid is inserted into a non-essential bacteriophage gene. In some embodiments, the non-essential bacteriophage gene is gp49, gp75, hoc, gp0.7, gp4.3, gp4.5, gp4.7, gp0.6, gp0.65, gp0.7, gp4.3, or gp4.5. In some embodiments, the target bacterium is C. difficile. In some embodiments, the temperate bacteriophage is ϕCD146 or ϕCD24-2. In some embodiments, the target bacterium is killed by the lytic activity of the temperate bacteriophage, by the activity of a CRISPR-Cas system using the first spacer sequence or the crRNA transcribed therefrom, or both. In some embodiments, the target bacterium is killed by the activity of the CRISPR-Cas system independently of the lytic activity of the temperate bacteriophage. In some embodiments, activity of the CRISPR-Cas system supplements or enhances lytic activity of the temperate bacteriophage. In some embodiments, lytic activity of the temperate bacteriophage and activity of the CRISPR-Cas system are synergistic. In some embodiments, lytic activity of the temperate bacteriophage, activity of the CRISPR-Cas system, or both is modulated by a concentration of the temperate bacteriophage. In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system, a Type II CRISPR-Cas system, or a Type III CRISPR-Cas system. In some embodiments, the CRISPR-Cas system is a Type I CRISPR-Cas system. In some embodiments, the temperate bacteriophage does not confer any new properties onto the target bacterium beyond cellular death caused by the lytic activity of the temperate bacteriophage, beyond the activity of the CRISPR-Cas array, or both. In some embodiments, disclosed herein is a method of treating a disease in an individual in need thereof, the method comprising administering the pharmaceutical composition disclosed herein. In some embodiments, the individual is a mammal. In some embodiments, the disease is a bacterial infection. In some embodiments, a bacterium causing the bacterial infection is an Escherichia coli, Salmonella enterica, Bacillus subtilis, Clostridium acetobutylicum, Clostridium ljungdahlii, Clostridioides difficile, Clostridium bolteae, Acinetobacter baumannii, Mycobacterium tuberculosis, Mycobacterium abscessus, Mycobacterium intracellulare, Mycobacterium fortuitum, Mycobacterium chelonae, Mycobacterium avium, Mycobacterium gordonae, Myxococcus xanthus, Streptococcus pyogenes, cyanobacteria, Staphylococcus aureus, methicillin resistant Staphylococcus aureus, Streptococcus pneumoniae, carbapenem-resistant Enterobacteriaceae, extended spectrum beta-lactamase (ESBL)-producing Enterobacteriaceae, Staphylococcus epidermidis, Staphylococcus salivarius, Corynebacterium minutissimum, Corynebacterium pseudodiphtheriticum, Corynebacterium striatum, Corynebacterium group G1, Corynebacterium group G2, Streptococcus mitis, Streptococcus sanguinis, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces israelii., Porphyromonas gingivalis., Prevotella melaninogenicus, Helicobacter pylori, Helicobacter felis, Enterococcus faecalis, Enterococcus faecium, Enterococcus gallinarum, Bacteroides fragilis, Bacteroides thetaiotaomicron, Fusobacterium nucleatum, Ruminococcus gnavus, or Campylobacter jejuni or any combination thereof. In some embodiments, the bacterium is a drug resistant bacterium that is resistant to at least one antibiotic. In some embodiments, the bacterium is a multi-drug resistant bacterium that is resistant to at least one antibiotic. In some embodiments, the at least one antibiotic comprises a cephalosporin, a fluoroquinolone, a carbapenem, a colistin, an aminoglycoside, vancomycin, streptomycin, or methicillin. In some embodiments the administering is intra-arterial, intravenous, intramuscular, oral, subcutaneous, inhalation, or any combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies conjugation efficiency of the Type I-B Clostridium difficile genome-targeting CRISPR array. The crRNA was cloned into pMTL84151 and conjugated into model C. difficile strains 630 and R20291. Viable transconjugants of the crRNA plasmid were recovered at approximately a 1-log lower frequency than that of the empty pMTL84151 plasmid control. Data are presented as the mean±standard error of the mean; *p<0.05, **p<0.01, ***p<0.001, ****p<0.0001, t-test Holm-Sidak method.

FIG. 2 is an overview of CRISPR phage engineering and mechanism of action. The genome of phage CD24-2 was modified to encode a bacterial-genome-targeting CRISPR array composed of a repeat-spacer-repeat meeting the requirements of the conserved C. difficile Type I-B system. The genome-targeting CRISPR array is transduced into the bacterial cell during infection and is expressed concurrently with the lytic genes of the bacteriophage. Cell death occurs by two independent mechanisms of action: irreparable genome damage by the natively expressed Type I-B Cas effector proteins directed by the CRISPR RNA, and cell lysis by the holin and endolysin expressed during lytic replication.

FIG. 3 is an exemplary transmission electron micrograph of wild type and CRISPR phage variants. Comparison of the wild type and crPhage morphology shows no differences in capsid size or sheath length.

FIG. 4A-FIG. 4C exemplify in vitro comparison of the activity and lysogen formation rates of wild type and engineered phage variants. FIG. 4A exemplifies time course of CFU reduction during in vitro infection by bacteriophage at an input MOI of 0.1. CRISPR-engineered phage offers an improvement in CFU reduction between 2 and 6 hours, but by 24 hours, all phage treated cultures recover. There was no observable effect of lysogeny gene knockout on the activity of the phage. Data are presented as the mean±standard error of the mean. FIG. 4B exemplifies time course of PCR-based detection of lysogeny in surviving bacterial colonies after phage infection. The CRISPR-enhanced phage exhibits impaired lysogen formation. Phage variants lacking key lysogeny genes exhibit no detectable lysogeny in vitro. FIG. 4C exemplifies dependency of CFU reduction on input MOI of phage variants. MOI ≥1 favors a rapid decrease in CFUs, but results are in rebound by 24 hours. By contrast, MOI ≤0.01 results in moderate CFU reductions that continue to decrease up to 24 hours. Data is presented as the mean±standard error of the mean; *p<0.05, **p<0.01, ***p<0.001, ****p<0.0001.

FIG. 5A-FIG. 5D exemplify C. difficile CD19 colonizes and causes disease in cefoperazone treated mice. FIG. 5A is an exemplary schematic depicting the experimental design and timeline; n=16 mice. FIG. 5B exemplifies the change in weight over time in mice challenged with C. difficile CD19. FIG. 5C exemplifies the total CFUs and spore CFUs in cecal content from infected mice at days 4 and 9 post challenge (n=8 per day). FIG. 5D exemplifies total histological score for cecal tissue from uninfected and infected mice from day 4 post challenge (n=8 per day). For FIG. 5B and FIG. 5C, data are presented as the mean±standard error of the mean; *p<0.05, **p<0.01, ***p<0.001, ****p<0.0001, Kruskal-Wallis One-Way ANOVA with Dunn's correction for multiple comparisons (for FIG. 5B), and Two-tailed Mann-Whitney for FIG. 5D.

FIG. 6A-FIG. 6L exemplify bacteriophage encoding a CRISPR targeting the C. difficile genome reduces C. difficile burden and clinical signs of disease in vivo. FIG. 6A is an exemplary schematic depicting experimental design, timeline, and treatment groups. Vehicle, wtPhage, and crPhage (n=20 mice over three experiments); wtPhage Δlys and crPhage Δlys (n=8 mice). FIG. 6B exemplifies fecal C. difficile vegetative CFUs from mice in each treatment group at days two and four post challenge. For day 2: vehicle n=13, wtPhage n=12, crPhage n=13, wtPhage Δlys and crPhage Δlys n=8. For day 4: vehicle n=12, wtPhage n=10, wtPhage Δlys n=2, crPhage Δlys n=8. FIG. 6C exemplifies the vegetative C. difficile CFUs from cecal content harvested four days post challenge. Vehicle, wtPhage n=10, crPhage n=9, wtPhage Δlys and crPhage Δlys n=8. FIG. 6D exemplifies total histologic score for cecal tissue harvested four days post challenge. Each variable has a scale from 0-4, maximal histologic summary score is 12. Uninfected vehicle, wtPhage Δlys and crPhage Δlys n=8; vehicle, wtPhage, and crPhage n=4. FIG. 6E illustrates representative images of cecal tissue harvested from mice of each treatment group on day four post challenge. Scale bar is 500 μm. FIG. 6F exemplifies total histologic score of colons harvested at day 4 post challenge. An uninfected, vehicle-treated group was included to control for the effects of the vehicle on colonic tissue. Uninfected vehicle, wtPhage Δlys and crPhage Δlys n=8; vehicle, wtPhage, and crPhage n=4. Each variable is scored on a scale of 0-4, thus the total summary score for maximum tissue damage is 12. FIG. 6G illustrates representative images of hematoxylin and eosin stained colonic tissue from day 4 post challenge in FIG. 6F. Scale bar is 500 μm. FIG. 6H exemplifies the change in weight in mice from each treatment group at four days post challenge. Uninfected vehicle n=4, vehicle, wtPhage, and crPhage n=14, and both wtPhage Δlys and crPhage Δlys n=8. FIG. 6I exemplifies percent of lysogens isolated from feces of mice treated with either wtPhage Δlys or the crPhage Δlys over the course of the experiment (n=8 mice per day, with 6 colonies per mouse screened by PCR). FIG. 6J exemplifies toxin gene expression in C. difficile CD19 and CD19 lysogenized with wtPhage. Results are from n=3 biological replicates for each strain at each time point. FIG. 6K exemplifies the PFUs from fecal content at two and four days post challenge. All treatments n=8-12 fecal samples were tested except for wtPhage Δlys (n=2) at day 4 due to lack of defecation. FIG. 6L exemplifies that crPhage lysogenizes more slowly in vivo than wtPhage.

For FIG. 6B, FIG. 6F, and FIG. 6J data are presented as the mean±standard error of the mean; *p<0.05, **p<0.01, ***p<0.001, ****p<0.0001, Kruskal-Wallis One-Way ANOVA with Dunn's correction for multiple comparisons, and Geisser-Greenhouse Two-Way ANOVA with Sidak's correction for multiple comparisons for FIG. 6J. For FIG. 611, FIG. 6C, FIG. 6D, and FIG. 6K data are presented as the mean±standard error of the mean; *p<0.05, **p<0.01, ***p<0.001, ****p<0.0001, Kruskal-Wallis One-Way ANOVA with Dunn's correction for multiple comparisons (for FIG. 611, FIG. 6C, FIG. 6D, and FIG. 6K).

FIG. 7A-FIG. 7C exemplify in vitro comparison of the activity and lysogen formation rates of wild type (WT) and engineered phage variant (WT ΔcI). FIG. 7A illustrates the region of the genome that was removed in this example. FIG. 7B illustrates time course of CFU reduction during in vitro infection by bacteriophage. FIG. 7C illustrates time course of PCR-based detection of lysogeny in surviving bacterial colonies after phage infection. The engineered phage variant did not affect lysogeny formation rate or phage kill.

FIG. 8A-FIG. 8C exemplify in vitro comparison of the activity and lysogen formation rates of wild type (WT) and engineered phage variant (WT ΔcI). FIG. 8A illustrates the region of the phage genome that was removed in this example. FIG. 8B illustrates time course of CFU reduction during in vitro infection by bacteriophage. FIG. 8C illustrates time course of PCR-based detection of lysogeny in surviving bacterial colonies after phage infection. The engineered phage variant slightly decreases lysogeny formation rate but does not improve phage kill.

FIG. 9A-FIG. 9D exemplify in vitro comparison of the activity and lysogen formation rates of wild type (WT) and engineered phage variants. FIG. 9A illustrates the region of the phage genome that was removed in this example. FIG. 9B illustrates time course of CFU reduction during in vitro infection by bacteriophage. FIG. 9C illustrates time course of PCR-based detection of lysogeny in surviving bacterial colonies after phage infection. The engineered phage variant (WT ΔcI) significantly slows lysogeny formation rate and improves phage kill. FIG. 9D exemplifies that lysogeny module knockout coupled with CRISPR RNA (gp75×1249) prevents lysogeny.

FIG. 10A-FIG. 10E exemplify in vitro comparison of the activity and lysogen formation rates of wild type (WT) and engineered phage variants (gp75—CRISPR enhanced; WT ΔcI—lysogeny knock out; gp75 ΔcI—CRISPR enhanced lysogeny knock out). FIG. 10A illustrates the design of counter selective crRNA targeting the lysogeny region. FIG. 10B and FIG. 10D illustrate time course of CFU reduction during in vitro infection by bacteriophage. FIG. 10C and FIG. 10E illustrate time course of PCR-based detection of lysogeny in surviving bacterial colonies after phage infection. The engineered phage variant (WT ΔcI) abolishes lysogeny formation rate and improves phage kill. Further, the addition of CRISPR to lysogeny knock out further improved phage kill.

FIGS. 11A-11C depict the effects of deletion of the predicted lysogeny region in phage p1473. FIG. 11A depicts the predicted lysogeny region of p1473. FIG. 11B depicts the dilution series of a wildtype p14′73, Var002, Var006, Var010, and Var012 plated on a lawn of Staphylococcus aureus. FIG. 11C depicts a close up image showing the larger plaque morphology for wildtype-1473, Var010, and Var012.

### DETAILED DESCRIPTION

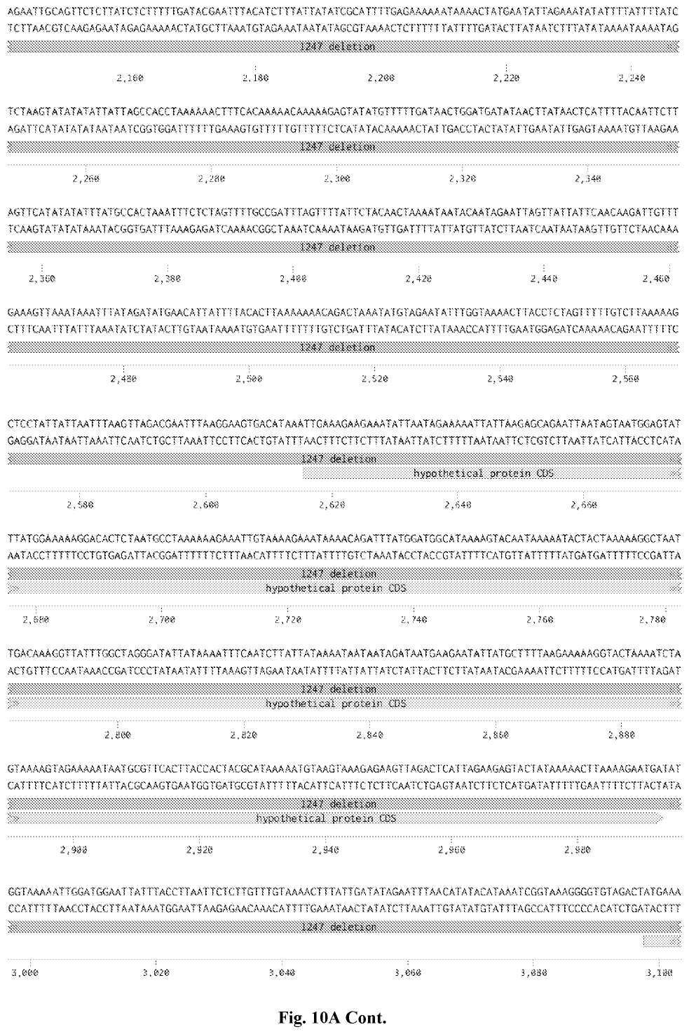

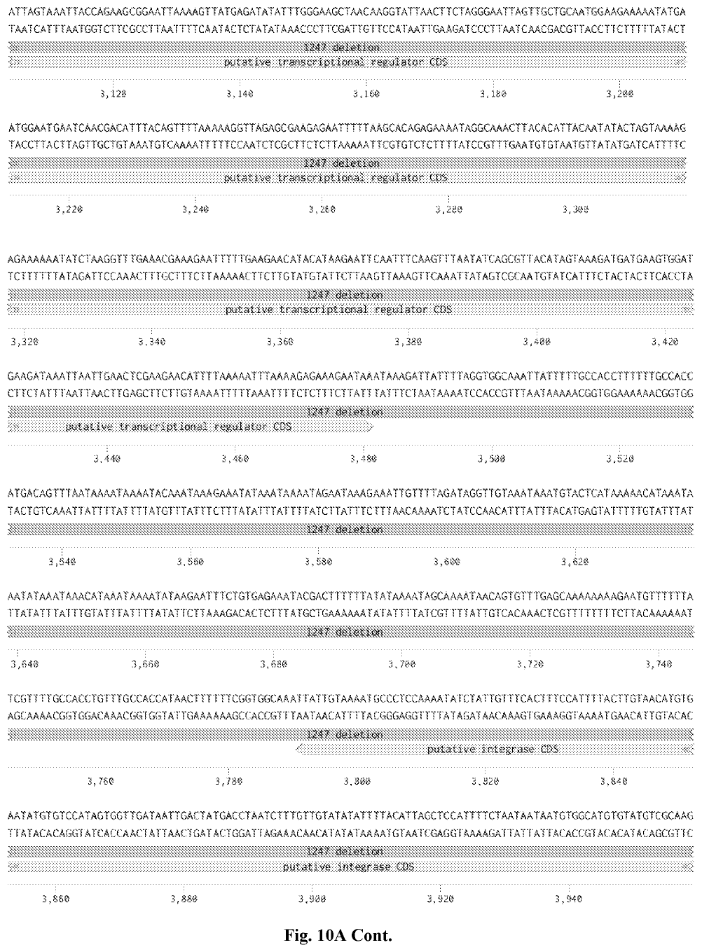

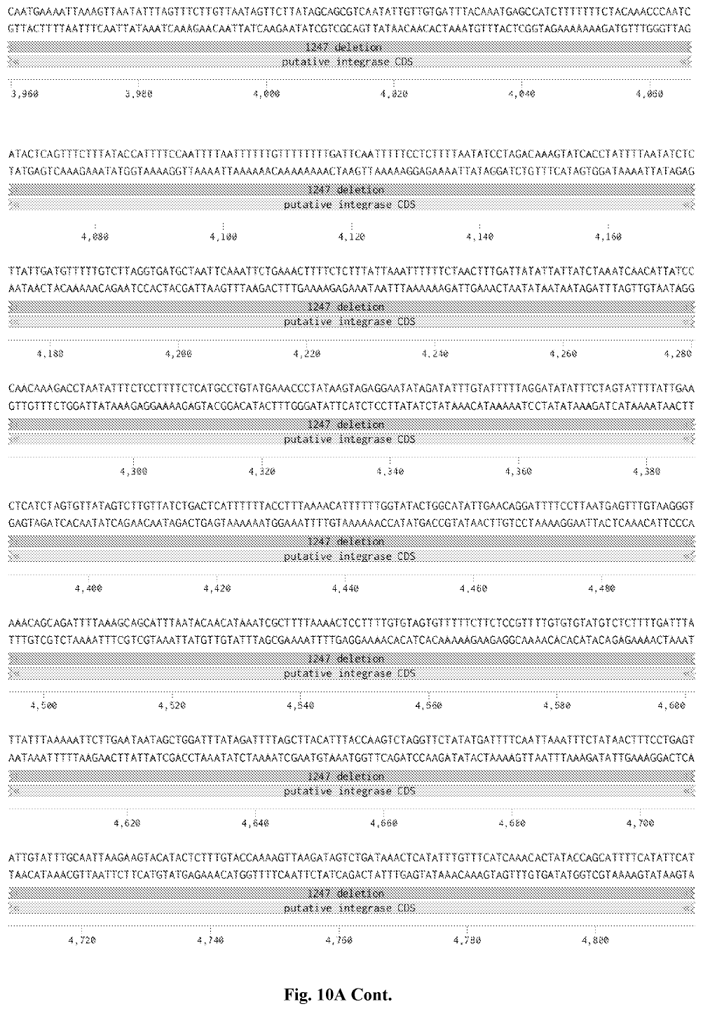

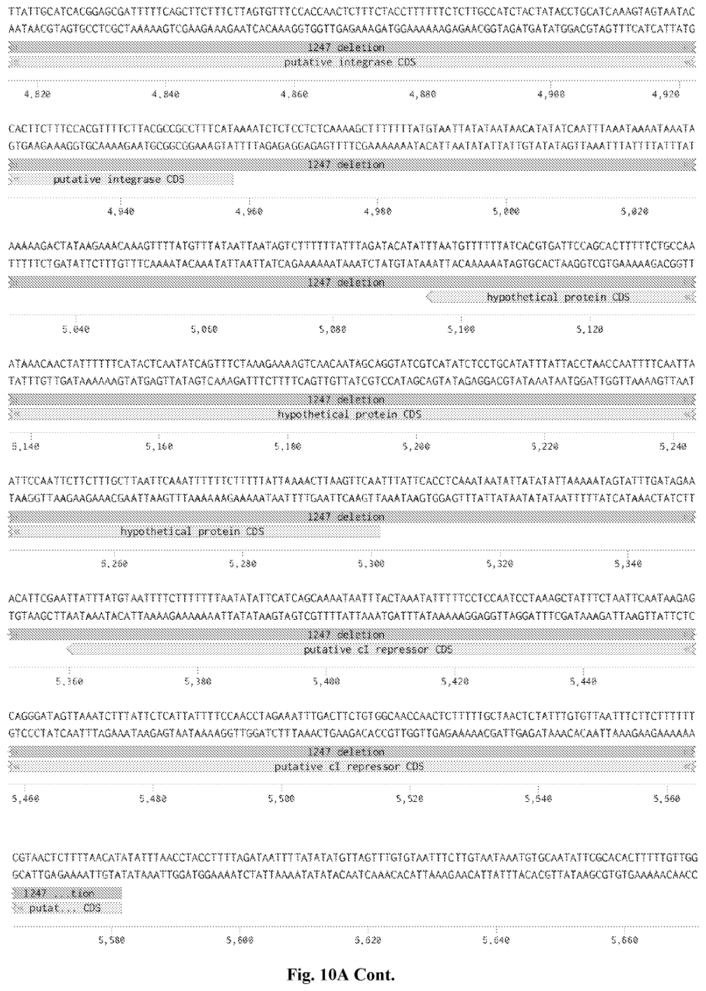

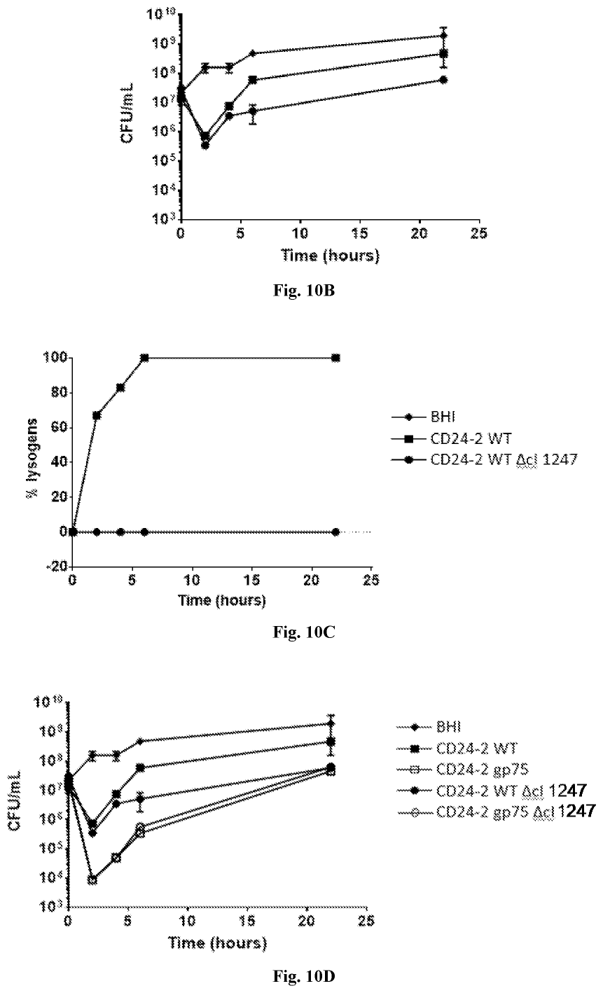

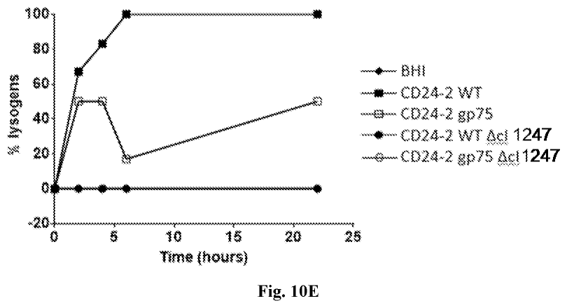

Disclosed herein, in certain embodiments, are bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. Further disclosed herein, in certain embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the region annotated as “1247 deletion” (also referred to as a 1247cI deletion) in FIG. 10a. Disclosed herein, in certain embodiments, are pharmaceutical composition comprising: (a) a bacteriophage disclosed herein, or a temperate bacteriophage disclosed herein; and (b) a pharmaceutically acceptable excipient. Further disclosed herein, in certain embodiments, are methods for killing a target bacterium, the method comprising introducing into the target bacterium a temperate bacteriophage comprising a first nucleic acid sequence encoding a first spacer sequence or a crRNA transcribed therefrom, wherein the first spacer sequence is complementary to a target nucleotide sequence from a target gene in the target bacterium, provided that the bacteriophage is rendered lytic by removal of the region annotated as “1247 deletion” in FIG. 10a, thereby killing the target bacterium. Also disclosed herein, in certain embodiments, are methods of treating a disease in an individual in need thereof, the method comprising administering a pharmaceutical composition disclosed herein.

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology. Likewise, one of skill in the art will also understand the interchangeability of terms designating the various anti-CRISPR proteins due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

As used herein, “chimeric” refers to a nucleic acid molecule or a polypeptide in which at least two components are derived from different sources (e.g., different organisms, different coding regions).

“Complement” as used herein means 100% complementarity or identity with the comparator nucleotide sequence or it means less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, functional elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, a “target nucleotide sequence” refers to the portion of a target gene that is complementary to the spacer sequence of the recombinant CRISPR array.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCG, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9).

As used herein, type I Clustered Regularly Interspaced Short Palindromic Repeats (CRISPR)-associated complex for antiviral defense (Cascade) refers to a complex of polypeptides involved in processing of pre-crRNAs and subsequent binding to the target DNA in type I CRISPR-Cas systems. These polypeptides include, but are not limited to, the Cascade polypeptides of type I subtypes I-A, I-B, I-C, I-D, I-E, I-F, and I-U. Non-limiting examples of type I-A polypeptides include Cas7 (Csa2), Cas8a1 (Csx13), Cas8a2 (Csx9), Cas5, Csa5, Cas6a, Cas3′ and/or a Cas3″. Non-limiting examples of type I-B polypeptides include Cas6b, Cas8b (Csh1), Cas7 (Csh2) and/or Cas5. Non-limiting examples of type I-C polypeptides include Cas5d, Cas8c (Csd1), and/or Cas7 (Csd2). Non-limiting examples of type I-D polypeptides include Cas10d (Csc3), Csc2, Csc1, and/or Cas6d. Non-limiting examples of type I-E polypeptides include Cse1 (CasA), Cse2 (CasB), Cas7 (CasC), Cas5 (CasD) and/or Cas6e (CasE). Non-limiting examples of type I-F polypeptides include Cys1, Cys2, Cas7 (Cys3) and/or Cas6f (Csy4). In some embodiments, a recombinant nucleic acid described herein comprises, consists essentially of, or consists of, a nucleotide sequence encoding a subset of type-I Cascade polypeptides that function to process a CRISPR array and subsequently bind to a target DNA using the spacer of the processed CRISPR RNA as a guide.

A “CRISPR array” as used herein means a nucleic acid molecule that comprises at least two repeat sequences, or a portion of each of said repeat sequences, and at least one spacer sequence. One of the two repeat sequences, or a portion thereof, is linked to the 5′ end of the spacer sequence and the other of the two repeat sequences, or portion thereof, is linked to the 3′ end of the spacer sequence. In a recombinant CRISPR array, the combination of repeat sequences and spacer sequences is synthetic, made by man and not found in nature. In some embodiments, a “CRISPR array” refers to a nucleic acid construct that comprises from 5′ to 3′ at least one repeat-spacer sequences (e.g., about 1, 2, 3, 4, 5, 6, 7, 8, 9, 10, 11, 12, 13, 14, 15, 16, 17, 18, 19, 20, 21, 22, 23, 24, 25 or more repeat-spacer sequences, and any range or value therein), wherein the 3′ end of the 3′ most repeat-spacer sequence of the array are linked to a repeat sequence, thereby all spacers in said array are flanked on both the 5′ end and the 3′ end by a repeat sequence.

As used herein, “spacer sequence” or “spacer” refers to a nucleotide sequence that is complementary to a target DNA (i.e., target region in the genome or the “protospacer sequence,” which is adjacent to a protospacer adjacent motif (PAM) sequence). The spacer sequence is fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target DNA.

A “repeat sequence” as used herein, refers to, for example, any repeat sequence of a wild-type CRISPR locus or a repeat sequence of a synthetic CRISPR array that are separated by “spacer sequences” (e.g., a repeat-spacer-repeat sequence). A repeat sequence useful with this disclosure is any known or later identified repeat sequence of a CRISPR locus or it is a synthetic repeat designed to function in a CRISPR system, for example CRISPR Type I system.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

In some embodiments, the recombinant nucleic acid molecules, nucleotide sequences and polypeptides disclosed herein are “isolated.” An “isolated” nucleic acid molecule, an “isolated” nucleotide sequence or an “isolated” polypeptide is a nucleic acid molecule, nucleotide sequence or polypeptide that exists apart from its native environment. In some instances, an isolated nucleic acid molecule, nucleotide sequence or polypeptide exists in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95% pure, or purer.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

As used herein the term “biofilm” means an accumulation of microorganisms embedded in a matrix of polysaccharide. Biofilms form on solid biological or non-biological surfaces, as well as at liquid-air interfaces, and are medically important, accounting for over 80 percent of microbial infections in the body.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, and mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab). Mollusk subjects include but are not limited to species used in aquaculture (e.g., abalone, mussel, oyster, clams, scallop). In some embodiments, suitable subjects include both males and females and subjects of any age, including embryonic (e.g., in-utero or in-ovo), infant, juvenile, adolescent, adult and geriatric subjects. In some embodiments, a subject is a human.”

As used herein, the term “isolated: in context of a nucleic acid sequence is a nucleic acid sequence that exists apart from its native environment.

As used herein, “pharmaceutically acceptable” means a material that is not biologically or otherwise undesirable, i.e., the materials are administered to a subject without causing any undesirable biological effects such as toxicity.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type III, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-C CRISPR-Cas system derived from Pseudomonas aeruginosa. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system. In some embodiments, the CRISPR-Cas system is a Type II CRISPR-Cas system. In some embodiments, the CRISPR-Cas system is a Type III CRISPR-Cas system.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

In some embodiments, the Type I CRISPR-Cas system is a Type I-A system, Type I-B system, Type I-C system, Type I-D system, Type I-E system, or Type I-F system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (b) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (c) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); (d) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F); (e) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); and/or (f) a nucleotide sequence encoding a Cas10d (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D). In some embodiments, the Type I Cascade complex comprises a Cascade polypeptide disclosed herein

### CRISPR Array

In some embodiments, the CRISPR array (crArray) disclosed herein comprises a spacer sequence and at least one repeat sequence. In some embodiments, the CRISPR array encodes a processed, mature crRNA. In some embodiments, the mature crRNA is introduced into a phage or a target bacterium described herein. In some embodiments, the phage comprises a nucleic acid that encodes a processed, mature crRNA. In some embodiments, an endogenous or exogenous Cas6 processes the CRISPR array into mature crRNA. In some embodiments, an exogenous Cas6 is introduced into the phage. In some embodiments, the phage comprises an exogenous Cas6. In some embodiments, an exogenous Cas6 is introduced into a target bacterium.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and (3) processing of the pre-crRNA by Cascade or members of Cascade, such as Cash, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

In some embodiments, the CRISPR array comprises a spacer sequence. In some embodiments, the CRISPR array further comprises at least one repeat sequence. In some embodiments, the at least one repeat sequence is operably linked to the spacer sequence at either its 5′ end or its 3′ end. In some embodiments, a CRISPR array is of any length and comprises any number of spacer nucleotide sequences alternating with repeat nucleotide sequences necessary to achieve the desired level of killing of a target bacterium by targeting one or more target sequences. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array as disclosed herein, comprises essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

### Spacer Sequence

In some embodiments, the spacer sequence is complementary to a target nucleotide sequence in a target bacterium. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5 ‘ region of the spacer sequence is 100% complementary to a target nucleotide sequence while the 3’ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about 30 to 39 nucleotides, about 31 to about 38 nucleotides, about 32 to about 37 nucleotides, about 33 to about 36 nucleotides, about 34 to about 35 nucleotides, or about 35 nucleotides In some embodiments, the P. aeruginosa Type I-C Cas system has a spacer length of about 34 nucleotides. In some embodiments, the P. aeruginosa Type I-C Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different but are complementary to one or more target nucleotide sequences. In some embodiments, the identity of two or more spacer sequences of the CRISPR array is different and are complementary to one or more target nucleotide sequences that are overlapping sequences. In some embodiments, the identity of two or more spacer sequences of the CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif).

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9)

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, a non-essential gene is any gene of an organism that is not critical for survival. However, being non-essential is highly dependent on the circumstances in which an organism lives.

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI GenBank or the PATRIC database. NCBI GenBank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

### Repeat Nucleotide Sequences

In some embodiments, a repeat nucleotide sequence of the CRISPR array comprises a nucleotide sequence of any known repeat nucleotide sequence of a CRISPR-Cas system. In some embodiment, the CRISPR-Cas system is a Type I CRISPR-Cas system. In some embodiment, a repeat nucleotide sequence is of a synthetic sequence comprising the secondary structure of a native repeat from a Type I CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a repeat sequence found in Table 3. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

### Transcriptional Activators

In some embodiments, the nucleic acid sequence further comprises a transcriptional activator. In some embodiments, the transcriptional activator encoded regulates the expression of genes of interest within the target bacterium. In some embodiments, the transcriptional activator activates the expression of genes of interest within the target bacterium whether exogenous or endogenous. In some embodiments, the transcriptional activator activates the expression genes of interest within the target bacterium by disrupting the activity of one or more inhibitory elements within the target bacterium. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, any homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system. In E. coli and S. typhimurium, LeuO drives increased expression of the casABCDE operon which has predicted LeuO and H-NS binding sequences upstream of CasA.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, σA, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as endogenous CRISPR promoter, endogenous Cas operon promoter, p16, p1pp, or ptat. In some embodiments, the promoter is a phage promoter, such as the promoter for gp105 or gp245.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding components of a CRISPR-Cas system. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Bacteriophages

Disclosed herein, in certain embodiments, are temperate bacteriophages which have been rendered lytic, comprising a removal, replacement, or inactivation of a lysogeny region in the temperate bacteriophage, wherein the removal, replacement, or inactivation of a lysogeny region renders the temperate bacteriophage lytic.

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate phages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the phage stops replicating and stably resides within the host cell, either integrating not the bacterial genome or being maintained as an extrachromosomal plasmid. Temperate phages can also undergo lytic replication similar to their lytic bacteriophage counterparts. Whether a phage replicates lytically or undergoes lysogeny upon infection depends on a variety of factors including growth compositions and the physiological state of the cell. A bacterial cell that has a lysogenic phage integrated into its genome is referred to as a lysogenic bacterium or lysogen. Exposure to adverse conditions may trigger reactivation of the lysogenic phage, termination of the lysogenic state and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote, as in the case of integrase proteins that facilitate integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

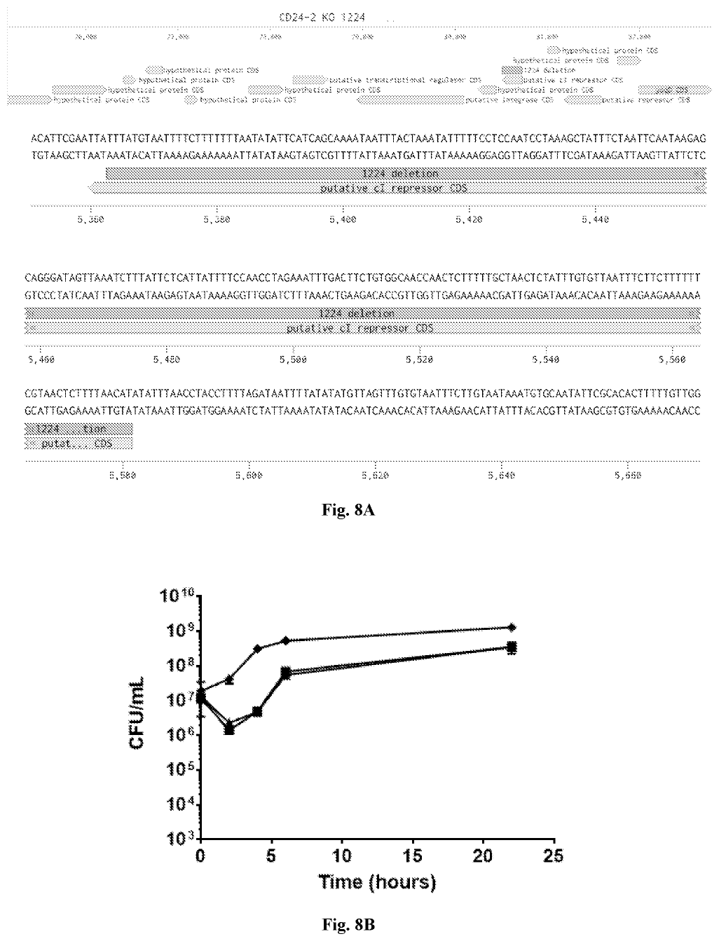

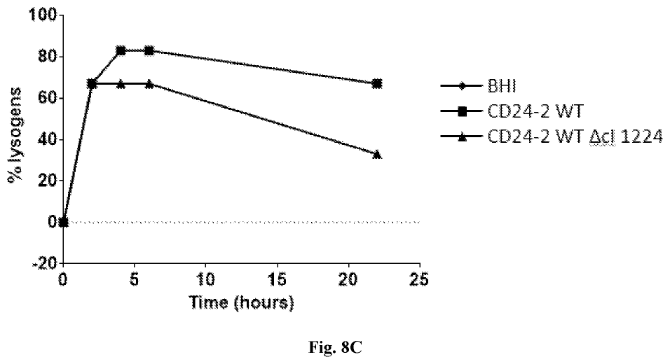

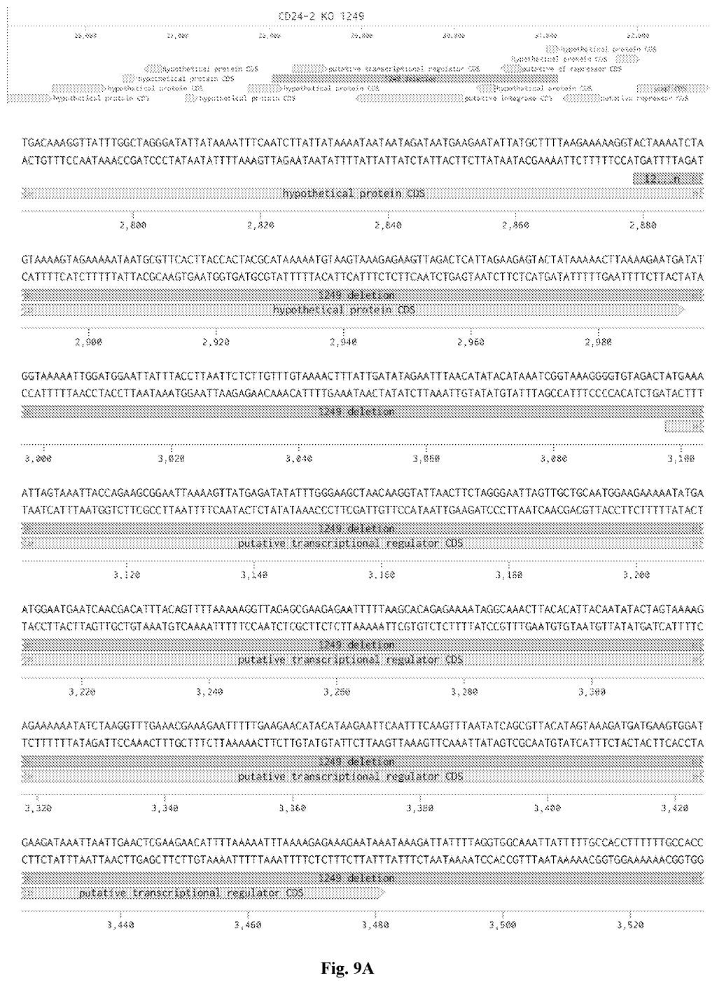

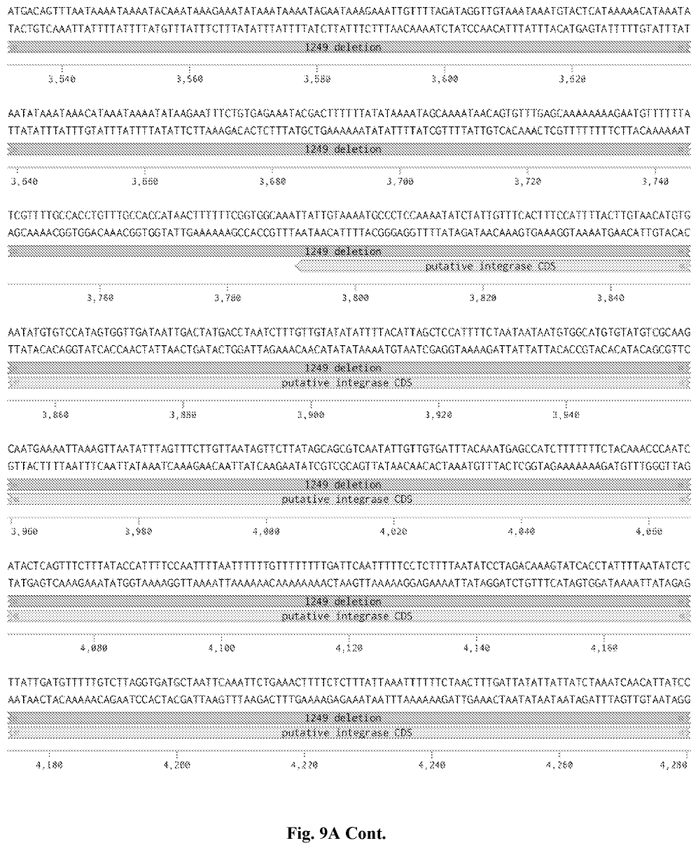

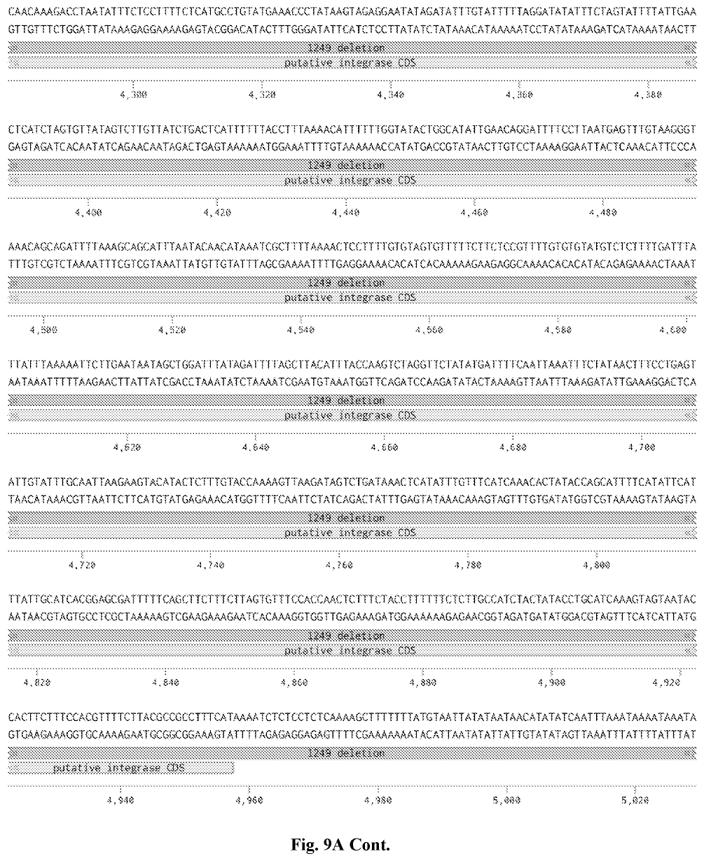

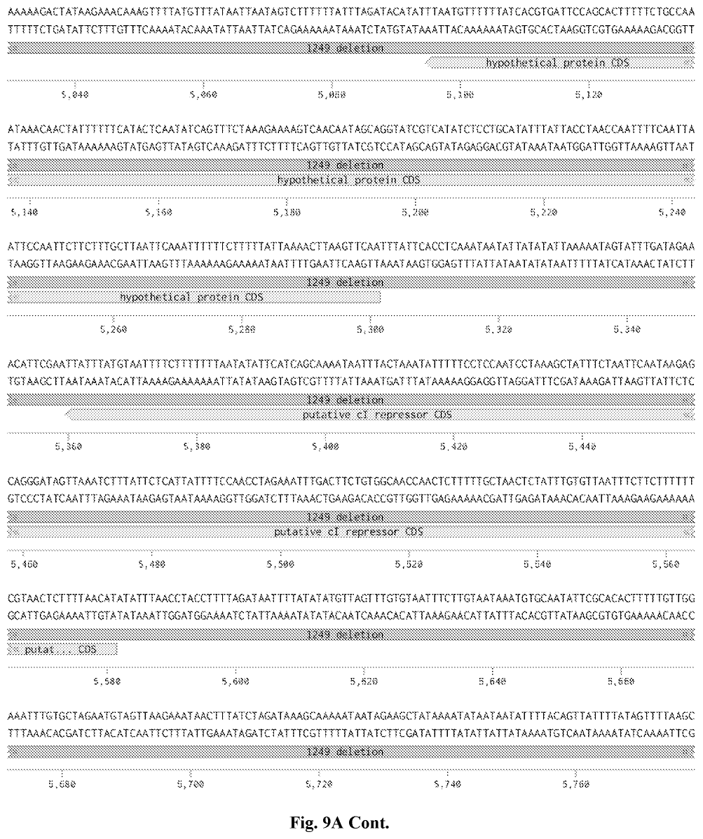

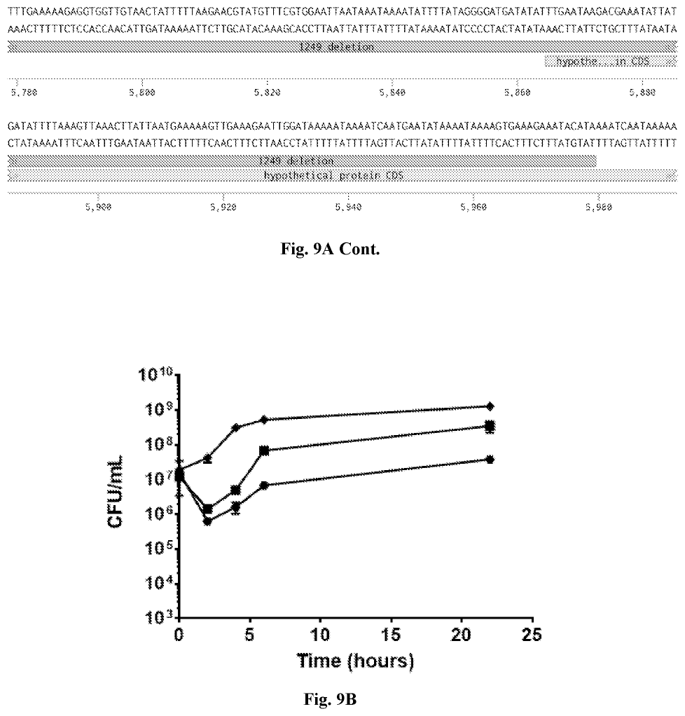

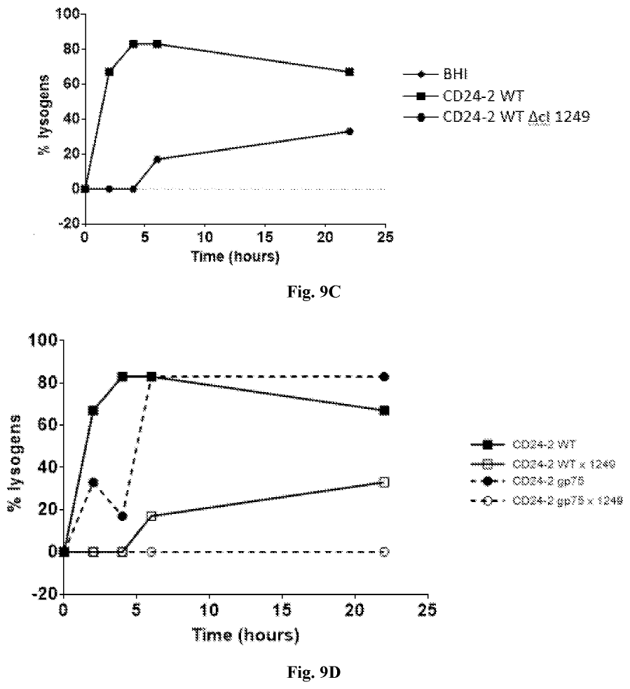

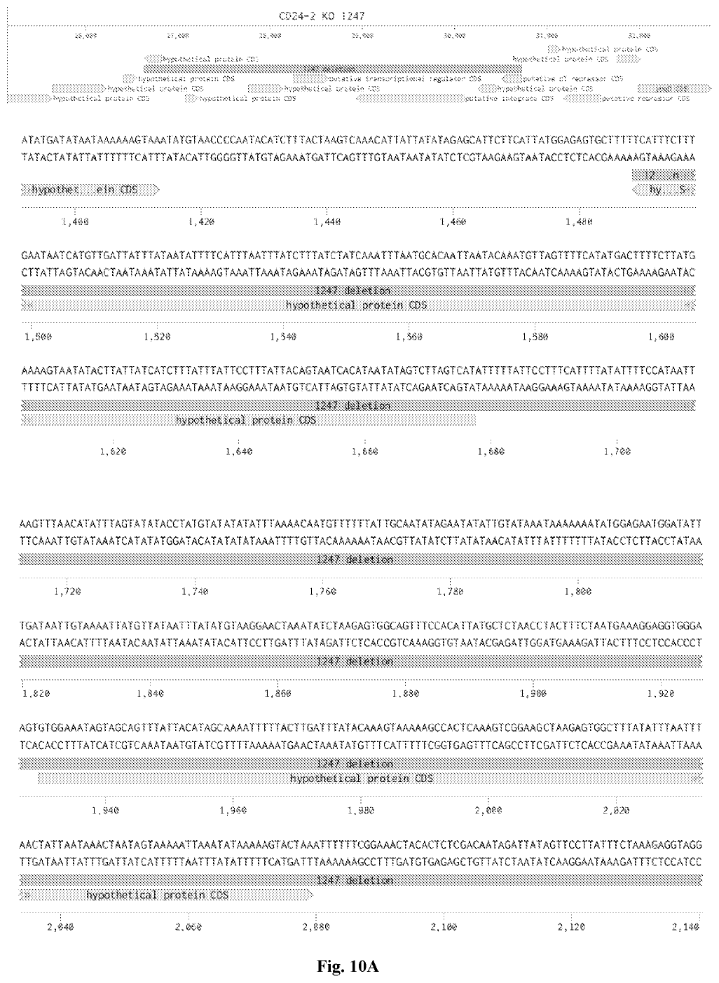

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by removal of the 1247 cI repressor region annotated as “1247 deletion” in FIG. 10a. In some embodiments, the bacteriophage is rendered lytic by removal of the 1249 cI repressor region annotated as “1249 deletion” in FIG. 9a. In some embodiments, the bacteriophage is rendered lytic by removal of the 1224 cI repressor region annotated as “1224 deletion” in FIG. 8a. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the first or second CRISPR array.

Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the region annotated as “1247 deletion” in FIG. 10a. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the target nucleotide sequence is in a non-essential gene or other genomic locus. In some embodiments, the target nucleotide sequence is in a non-essential gene. In some embodiments, the target nucleotide sequence is in a non-essential genomic locus. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the first nucleic acid sequence comprises a first CRISPR array further comprising at least one repeat sequence. In some embodiments, the at least one repeat sequence is operably linked to the first spacer sequence at either its 5′ end or its 3′ end. In some embodiments, the target bacterium is C. difficile.

In some embodiments, the bacteriophage or phagemid DNA is from a lysogenic or temperate bacteriophage. In some embodiments, the bacteriophages or phagemids include but are not limited to P1 phage, a λ, phage, a ϕC2 phage, a ϕCD27 phage, a ϕNM1 phage, Bc431 v3 phage, ϕ10 phage, ϕ25 phage, ϕ151 phage, A511-like phages, B054, 0176-like phages, or Campylobacter phages (such as NCTC 12676 and NCTC 12677). In some embodiments, the bacteriophage is ϕCD146 C. difficile bacteriophage.

In some embodiments, a plurality of bacteriophages are used together. In some embodiments, the plurality of bacteriophages used together targets the same or different bacteria within a sample or subject.

In some embodiments, bacteriophages of interest are obtained from environmental sources. or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system of a combination thereof. In some embodiments, the nucleic acid is inserted into the bacteriophage genome at a transcription terminator site at the end of an operon of interest. In some embodiments, the nucleic acid is inserted into a non-essential bacterial gene of other genomic locus. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is ϕCD146 C. difficile bacteriophage. In some embodiments, the bacteriophage is ϕCD24-2 C. difficile bacteriophage. In some embodiments, the bacteriophage is p1473 Staphylococcus aureus bacteriophage.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is gp49 from ϕCD146 C. difficile bacteriophage. In some embodiments, the non-essential gene to be removed and/or replaced from the bacteriophage is gp75 from ϕCD24-2 C. difficile bacteriophage. In some embodiments, the non-essential gene to be removed and/or replaced from the bacteriophage is a possible repressor and/or possible anti-repressor upstream of a possible integrase gene from p1473 Staphylococcus aureus bacteriophage

In some embodiments, the bacteriophage is a temperate bacteriophage with a lysogeny gene removed, replaced, or inactivated, thereby rendering the bacteriophage lytic. In some embodiments, the bacteriophage targets Pseudomonas spp. In some embodiments, the bacteriophage targets Pseudomonas aeruginosa. In some embodiments, the bacteriophage targets Escherichia spp. In some embodiments, the bacteriophage targets Escherichia coli. In some embodiments, the bacteriophage targets Staphylococcus spp. In some embodiments, the bacteriophage targets Staphylococcus aureus. In some embodiments, the bacteriophage targets Klebsiella spp. In some embodiments, the bacteriophage targets Klebsiella pneumoniae. In some embodiments, the bacteriophage targets Enterococcus spp. In some embodiments, the bacteriophage targets Enterococcus faecium. In some embodiments, the bacteriophage targets Enterococcus faecalis. In some embodiments, the bacteriophage targets Enterococcus gallinarum. In some embodiments, the bacteriophage targets Clostridioides spp. In some embodiments, the bacteriophage targets Clostridioides difficile. In some embodiments, the bacteriophage targets Bacteroides spp. In some embodiments, the bacteriophage targets Bacteroides fragilis. In some embodiments, the bacteriophage targets Bacteroides thetaiotaomicron. In some embodiments, the bacteriophage targets Fusobacterium spp. In some embodiments, the bacteriophage targets Fusobacterium nucleatum. In some embodiments, the bacteriophage targets Streptococcus spp. In some embodiments, the bacteriophage targets Streptococcus pneumoniae. In some embodiments, the bacteriophage targets Acinetobacter spp. In some embodiments, the bacteriophage targets Acinetobacter baumannii. In some embodiments, the bacteriophage targets Mycobacterium spp. In some embodiments, the bacteriophage targets Mycobacterium tuberculosis. In some embodiments, the bacteriophage targets Haemophilus spp. In some embodiments, the bacteriophage targets Haemophilus influenzae. In some embodiments, the bacteriophage targets Neisseria spp. In some embodiments, the bacteriophage targets Neisseria gonorrhoeae. In some embodiments, the bacteriophage targets Ruminococcus spp. In some embodiments, the bacteriophage targets Ruminococcus gnavus.

Disclosed herein, in certain embodiments, are bacteriophages comprising a complete exogenous CRISPR-Cas system. In some embodiments, the CRISPR-Cas system is Type I CRISPR-Cas system, Type II CRISPR-Cas system, Type III CRISPR-Cas system, Type IV CRISPR-Cas system, Type V CRISPR-Cas system, or Type VI CRISPR-Cas system. Disclosed herein, in certain embodiments, are bacteriophages comprising a nucleic acid sequence encoding a Type I CRISPR-Cas system comprising: (a) a CRISPR array; (b) a Cascade polypeptide; and (c) a Cas3 polypeptide.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle

### Methods of Killing a Target Bacterium

Disclosed herein, in certain embodiments, are methods of killing bacteria by introducing into the target bacterium any of the bacteriophages disclosed herein. In some embodiments, the method comprises introducing into a target bacterium a bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic, thereby killing the target bacterium. In some embodiments, the method comprises introducing into the target bacterium a temperate bacteriophage comprising a first nucleic acid sequence encoding a first spacer sequence or a crRNA transcribed therefrom, wherein the first spacer sequence is complementary to a target nucleotide sequence from a target gene in the target bacterium, provided that the bacteriophage is rendered lytic by removal of the region annotated as “1247 deletion” in FIG. 10a, thereby killing the target bacterium.

In some embodiments, the target bacterium is killed by the lytic activity of the temperate bacteriophage, by the activity of a CRISPR-Cas system using the first spacer sequence or the crRNA transcribed therefrom, or both. In some embodiments, the target bacterium is killed by the activity of the CRISPR-Cas system independently of the lytic activity of the temperate bacteriophage. In some embodiments, activity of the CRISPR-Cas system supplements or enhances lytic activity of the temperate bacteriophage. In some embodiments, lytic activity of the temperate bacteriophage and activity of the CRISPR-Cas system are synergistic. In some embodiments, lytic activity of the temperate bacteriophage, activity of the CRISPR-Cas system, or both is modulated by a concentration of the temperate bacteriophage relative to a concentration of the target bacterium. In some embodiments, the CRISPR-Cas system is endogenous to the target bacterium. In some embodiments, the CRISPR-Cas system is exogenous to the target bacterium.

In some embodiments, the CRISPR-Cas system is a Type I CRISPR-Cas system, a Type II CRISPR-Cas system, or a Type III CRISPR-Cas system. In some embodiments, the CRISPR-Cas system is a Type I CRISPR-Cas system. In some embodiments, the temperate bacteriophage does not confer any new properties onto the target bacterium beyond cellular death caused by the lytic activity of the temperate bacteriophage, beyond the activity of the CRISPR-Cas array, or both.

### Methods of Use

### Bacterial Infections

Disclosed herein, are methods of treating bacterial infections. In some embodiments, the bacteriophages disclosed herein treat or prevent diseases or conditions mediated or caused by bacteria as disclosed herein in a human or animal subjects. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, one or more target bacteria present in a bacterial population are pathogenic. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the pathogenic bacteria are various strains of C. difficile including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or 820291.

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject. In some embodiments, the target enteropathogenic bacterium is enteropathogenic E. coli (EPEC). In some embodiments, the bacteriophages are used to selectively modulate and/or kill one or more target diarrheagenic bacteria from a plurality of bacteria within the microbiome or gut flora of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome or gut flora of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, of a subject. In some embodiments, the bacteriophage disclosed herein are used to treat an infection, a disease, or a condition on a mucosal membrane of a subject. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), infections of the central nervous system, endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the bacteriophages are used to modulate and/or kill target bacteria on the mucosal membrane of a subject. In some embodiments, the bacteriophage treats acne and other related skin infections.

In some embodiments, the pathogenic bacteria are antibiotic resistant. In one embodiment, the pathogenic bacterium is methicillin-resistant Staphylococcus aureus (MRSA).

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the target bacterium comprises one or more species of the target bacterium. In some embodiments, the target bacterium comprises one or more strains of the target bacterium. In some embodiments, non-limiting examples of target bacteria include Escherichia spp., Salmonella spp., Bacillus spp., Corynebacterium spp., Clostridium spp., Clostridioides spp., Pseudomonas spp., Lactococcus spp., Acinetobacter spp., Mycobacterium spp., Myxococcus spp., Staphylococcus spp., Streptococcus spp., Enterococcus spp., Bacteroides spp., Fusobacterium spp., Actinomyces spp., Porphyromonas spp., or cyanobacteria. In some embodiments, non-limiting examples of bacteria include Escherichia coli, Salmonella enterica, Bacillus subtilis, Clostridium acetobutylicum, Clostridium ljungdahlii, Clostridioides difficile, Clostridium bolteae, Acinetobacter baumannii, Mycobacterium tuberculosis, Mycobacterium abscessus, Mycobacterium intracellulare, Mycobacterium fortuitum, Mycobacterium chelonae, Mycobacterium avium, Mycobacterium gordonae, Myxococcus xanthus, Streptococcus pyogenes, cyanobacteria, Staphylococcus aureus, methicillin resistant Staphylococcus aureus, Streptococcus pneumoniae, carbapenem-resistant Enterobacteriaceae, extended spectrum beta-lactamase (ESBL)-producing Enterobacteriaceae, Staphylococcus epidermidis, Staphylococcus salivarius, Corynebacterium minutissimum, Corynebacterium pseudodiphtheriticum, Corynebacterium striatum, Corynebacterium group G1, Corynebacterium group G2, Streptococcus mitis, Streptococcus sanguinis, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces israelii., Porphyromonas gingivalis., Prevotella melaninogenicus, Helicobacter pylori, Helicobacter felis, Enterococcus faecalis, Enterococcus faecium, Enterococcus gallinarum, Bacteroides fragilis, Bacteroides thetaiotaomicron, Fusobacterium nucleatum, Ruminococcus gnavus, or Campylobacter jejuni. Further non-limiting examples of bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Pseudomonas aeruginosa. In some embodiments, the bacterium is Escherichia coli. In some embodiments, the bacterium is Clostridioides difficile. In some embodiments, the bacterium is Staphylococcus aureus. In some embodiments, the bacterium is Klebsiella pneumoniae. In some embodiments, the bacterium is Enterococcus faecalis. In some embodiments, the bacterium is Enterococcus faecium. In some embodiments, the bacterium is Bacteroides fragilis. In some embodiments, the bacterium is Bacteroides thetaiotaomicron. In some embodiments, the bacterium is Fusobacterium nucleatum. In some embodiments, the bacterium is Enterococcus gallinarum. In some embodiments, the bacterium is Ruminococcus gnavus. In some embodiments, the bacterium is Acinetobacter baumannii. In some embodiments, the bacterium is Mycobacterium tuberculosis. In some embodiments, the bacterium is Streptococcus pneumoniae. In some embodiments, the bacterium is Haemophilus influenzae. In some embodiments, the bacterium is Neisseria gonorrhoeae.

In some embodiments, the target bacterium causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the target bacterium causes urinary tract infection. In some embodiments, the E. coli causes urinary tract infection. In some embodiments, the target bacterium causes and/or exacerbates an inflammatory disease. In some embodiments, the target bacterium causes and/or exacerbates an autoimmune disease. In some embodiments, the target bacterium causes and/or exacerbates inflammatory bowel disease (IBD). In some embodiments, the E. coli causes inflammatory bowel disease (IBD). In some embodiments, the target bacterium causes and/or exacerbates psoriasis. In some embodiments, the target bacterium causes and/or exacerbates psoriatic arthritis (PA). In some embodiments, the target bacterium causes and/or exacerbates rheumatoid arthritis (RA). In some embodiments, the target bacterium causes and/or exacerbates systemic lupus erythematosus (SLE). In some embodiments, the target bacterium causes and/or exacerbates multiple sclerosis (MS). In some embodiments, the target bacterium causes and/or exacerbates Graves' disease. In some embodiments, the target bacterium causes and/or exacerbates Hashimoto's thyroiditis. In some embodiments, the target bacterium causes and/or exacerbates Myasthenia gravis. In some embodiments, the target bacterium causes and/or exacerbates vasculitis. In some embodiments, the target bacterium causes and/or exacerbates cancer. In some embodiments, the target bacterium causes and/or exacerbates cancer progression. In some embodiments, the target bacterium causes and/or exacerbates cancer metastasis. In some embodiments, the target bacterium causes and/or exacerbates resistance to cancer therapy. In some embodiments, the therapy used to address cancer includes, but is not limited to, chemotherapy, immunotherapy, hormone therapy, targeted drug therapy, and/or radiation therapy. In some embodiments, the cancer develops in organs including, but not limited to the, anus, bladder, blood and blood components, bone, bone marrow, brain, breast, cervix uteri, colon and rectum, esophagus, kidney, larynx, lymphatic system, muscle (i.e., soft tissue), oral cavity and pharynx, ovary, pancreas, prostate, skin, small intestine, stomach, testis, thyroid, uterus, and/or vulva. In some embodiments, the target bacterium causes and/or exacerbates disorders of the central nervous system (CNS). In some embodiments, the target bacterium causes and/or exacerbates attention deficit/hyperactivity disorder (ADHD). In some embodiments, the target bacterium causes and/or exacerbates autism. In some embodiments, the target bacterium causes and/or exacerbates bipolar disorder. In some embodiments, the target bacterium causes and/or exacerbates major depressive disorder. In some embodiments, the target bacterium causes and/or exacerbates epilepsy. In some embodiments, the target bacterium causes and/or exacerbates neurodegenerative disorders including, but not limited to, Alzheimer's disease, Huntington's disease, and/or Parkinson's disease.

Cystic fibrosis and cystic fibrosis-associated bronchiectasis is associated with infection by Pseudomonas aeruginosa. See, e.g., P. Farrell, et al, Radiology, Vol. 252, No. 2, pp. 534-543 (2009). In some embodiments, one or more bacteriophage are administered to a patient with cystic fibrosis or cystic fibrosis-associated bronchiectasis. In some embodiments, a combination of two or more bacteriophage are administered to a patient with cystic fibrosis or cystic fibrosis-associated bronchiectasis. In some embodiments, administration of the bacteriophage to a patient with cystic fibrosis or cystic fibrosis-associated bronchiectasis results in a reduction in bacterial load in the patient. In some embodiments, the reduction in bacterial load results in a clinical improvement in the patient with cystic fibrosis or cystic fibrosis-associated bronchiectasis.

Non-cystic fibrosis bronchiectasis is associated with infection by Pseudomonas aeruginosa. See, e.g., R. Wilson, et al, Respiratory Medicine, Vol. 117, pp. 179-189 (2016). In some embodiments, one or more bacteriophage are administered to a patient with non-cystic fibrosis bronchiectasis. In some embodiments, a combination of two or more bacteriophage are administered to a patient with non-cystic fibrosis bronchiectasis. In some embodiments, administration of the bacteriophage to a patient with non-cystic fibrosis bronchiectasis results in a reduction in bacterial load in the patient. In some embodiments, the reduction in bacterial load results in a clinical improvement in the patient with non-cystic fibrosis bronchiectasis.

In some embodiments, a target bacterium is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, Ceftazidime, Cefepime, Piperacillin, Ticarcillin, Linezolid, a Streptogramin, Tigecycline, Daptomycin, or any combination thereof. Examples of MDR strains include: Vancomycin-Resistant Enterococci (VRE), Methicillin-Resistant Staphylococcus aureus (MRSA), Extended-spectrum β-lactamase (ESBL)-producing Gram-negative bacteria, Klebsiella pneumoniae carbapenemase (KPC)-producing Gram-negatives, Multidrug-Resistant gram-negative rods (MDR GNR), and MDRGN bacteria such as Enterobacter species, E. coli, Klebsiella pneumoniae, Acinetobacter baumannii, or Pseudomonas aeruginosa.

In some embodiments the target bacterium is Klebsiella pneumoniae. In some embodiments, the target bacterium is Staphylococcus aureus. In some embodiments, the target bacterium is Enterococcus. In some embodiments, the target bacterium is Acinetobacter. In some embodiments, the target bacterium is Pseudomonas. In some embodiments, the target bacterium is Enterobacter. In some embodiments, the target bacterium is Clostridium difficile. In some embodiments, the target bacterium is E. coli. In some embodiments, the target bacterium is Clostridium bolteae. In some embodiments, the methods and compositions disclosed herein are for use in veterinary and medical applications as well as research applications

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the bacteriophages are used to selectively modulate and/or kill one or more target enteropathogenic bacteria from a plurality of bacteria within the microbiome of a subject. In some embodiments, the target enteropathogenic bacterium is enteropathogenic E. coli (EPEC). In some embodiments, the bacteriophages are used to selectively modulate and/or kill one or more target diarrheagenic bacteria from a plurality of bacteria within the microbiome of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of Enterobacteriaceae, Pasteurellaceae, Fusobacteriaceae, Neisseriaceae, Veillonellaceae, Gemellaceae, Bacteriodales, Clostridiales, Erysipelotrichaceae, Bifidobacteriaceae, Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, Proteus, Escherichia Coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, and Coprococcus iomes.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiments, microbiome-related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera such as Pseudomonas which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiments, a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiments, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or is transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is used in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products may be sanitized with bacteriophage. For example, freshly-cut produce frequently arrives at the processing plant contaminated with pathogenic bacteria. This has led to outbreaks of food-borne illness traceable to produce. In some embodiments, the application of bacteriophage preparations to agricultural produces substantially reduces or eliminates the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, or produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, taking several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial or archaeal infections or to disinfect an area. In some embodiments, the pharmaceutical composition comprises any of the reagents discussed above in a pharmaceutically acceptable carrier. In some embodiments, a pharmaceutical composition or method disclosed herein treats lung infections (e.g. cystic-fibrosis associated pneumonia (CFP), non-cystic-fibrosis associated bronchiectasis (NCFB), hospital-associated pneumonia (HAP), ventilator-associated pneumonia (VAP)), systemic infections (e.g. bacteremia, skin and soft tissue infections (SSSI)), GI microbiome dysbiosis (CDI), urinary tract infections (e.g. chronic urinary tract infections (cUTI)), and/or inflammatory diseases (e.g. inflammatory bowel disease (IBD), Crohn' Disease, ulcerative colitis).

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, during the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiments, a method is described of treating subjects in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition are produced with any solid particulate medicament aerosol generator, by techniques known in the pharmaceutical art.

In some embodiments, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and include but are not limited to solvents, dispersion media, diluents, or other liquid vehicles, dispersion or suspension aids, surface active agents, isotonic agents, thickening or emulsifying agents, preservatives, solid binders, and lubricants. Non-limiting examples of suitable excipients include but are not limited to a buffering agent, a preservative, a stabilizer, a binder, a compaction agent, a lubricant, a chelator, a dispersion enhancer, a disintegration agent, a flavoring agent, a sweetener, a coloring agent. In some embodiments, an excipient is a buffering agent. Non-limiting examples of suitable buffering agents include but are not limited to sodium citrate, magnesium carbonate, magnesium bicarbonate, calcium carbonate, and calcium bicarbonate.

In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but are not limited to antioxidants, such as alpha-tocopherol and ascorbate, and antimicrobials, such as parabens, chlorobutanol, and phenol. In some embodiments, antioxidants include but are not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, xylitol, and the like.

In some instances, a pharmaceutical formulation comprises a coloring agent. Non-limiting examples of suitable coloring agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Lincomycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### Dose

Dose and duration of the administration of a composition disclosed herein will depend on a variety of factors, including the subject's age, subject's weight, and tolerance of the phage. In some embodiments, a bacteriophage disclosed herein is administered to patients by oral administration. In some embodiments, a dose of phage between 10³ and 10²⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹ PFU. In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Kits

Disclosed herein are kits for use. In some embodiments, the kit comprises the nucleic acid constructs for the CRISPR arrays, as well as the bacteriophages and/or any other vectors/expression cassettes disclosed herein in a form suitable for introduction into a cell and/or administration to a subject. In some embodiments, the kit comprises other therapeutic agents, carriers, buffers, containers, devices for administration, and the like. In some embodiments, the kit comprises labels and/or instructions for repression of expression of a target gene and/or modulation of repression of expression of a target gene. In some embodiments, labeling and/or instructions includes, for example, information concerning the amount, frequency and method of introduction and/or administration of the nucleic acid constructs for the CRISPR arrays, transcriptional activators, and anti-CRISPR polypeptides, as well as the bacteriophages and/or any other vectors/expression cassettes.

In some embodiments, a kit for the killing of one target bacterium is provided, said kit comprising, consisting essentially of, consisting of nucleic acid constructs for the CRISPR arrays, transcriptional activators, and/or anti-CRISPR polypeptides, as well as the bacteriophages and/or any other vectors/expression cassettes necessary to achieve killing of the target bacteria by any embodiment disclosed herein.

In some embodiments, a kit for modulating the activity of a CRISPR-Cas system in a target bacterium is provided, the kit comprising, consisting essentially of, consisting of nucleic acid constructs for the CRISPR arrays, transcriptional activators, and anti-CRISPR polypeptides, as well as the bacteriophages and/or any other vectors/expression cassettes necessary to achieve modulation of a CRISPR-Cas system in a target bacteria by any embodiment disclosed herein.

In some embodiments, the nucleic acid constructs for the CRISPR arrays, transcriptional activators, and/or anti-CRISPR polypeptides of said kits are comprised on a single vector or expression cassette or on separate vectors or expression cassettes or within a single bacteriophage or a plurality of bacteriophages. In some embodiments, a kit comprises one or more bacteriophage disclosed herein. In some embodiments, the kits comprise instructions for use. In some embodiments, the instructions for practicing the methods are recorded on a suitable recording medium. In some embodiments, the instructions are printed on a substrate, such as paper or plastic, etc. In some embodiments, the instructions are present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source (e.g. via the Internet), are provided. In some embodiments, the kit includes a web address where the instructions are viewed and/or from which the instructions are downloaded.

Certain embodiments disclosed herein, both in their methods and compositions, will now are described with reference to the following examples. It should be appreciated that these examples are not intended to limit the scope of the claims to the disclosure, but are rather intended to be exemplary of certain embodiments. Any variations in the exemplified methods that occur to the skilled artisan are intended to fall within the scope of the disclosure.

### Numbered Embodiments

Numbered embodiment 1 comprises a bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. Numbered embodiment 2 comprises the bacteriophage of embodiment 1, wherein the bacteriophage is derived from a temperate bacteriophage. Numbered embodiment 3 comprises the bacteriophage of any one of embodiments 1-2, wherein the bacteriophage is rendered lytic by removal, replacement, or inactivation of a lysogenic gene. Numbered embodiment 4 comprises the bacteriophage of any one of embodiments 1-3, wherein the bacteriophage is rendered lytic by removal of a 1247 cI repressor region. Numbered embodiment 5 comprises the bacteriophage of any one of embodiments 1-4, wherein the bacteriophage is rendered lytic by the removal of a 1249 cI repressor region. Numbered embodiment 6 comprises the bacteriophage of any one of embodiments 1-5, wherein the bacteriophage is rendered lytic by the removal of a 1224 cI repressor region. Numbered embodiment 7 comprises the bacteriophage of any one of embodiments 1-6, wherein the bacteriophage is rendered lytic by the removal of a regulatory element of a lysogeny gene. Numbered embodiment 8 comprises the bacteriophage of any one of embodiments 1-7, wherein the bacteriophage is rendered lytic by the removal, alteration or replacement of a promoter of a lysogeny gene. Numbered embodiment 9 comprises the bacteriophage of any one of embodiments 1-8, wherein the bacteriophage is rendered lytic by the removal of a functional element of a lysogeny gene. Numbered embodiment 10 comprises the bacteriophage of any one of embodiments 1-9, wherein the bacteriophage is rendered lytic via a second CRISPR array comprising a second spacer directed to a lysogenic gene. Numbered embodiment 11 comprises the bacteriophage of any one of embodiments 1-10, wherein the bacteriophage infects multiple bacterial strains. Numbered embodiment 12 comprises the bacteriophage of any one of embodiments 1-11, wherein the target nucleotide sequence comprises all or a part of a promoter sequence for the target gene. Numbered embodiment 13 comprises the bacteriophage of any one of embodiments 1-12, wherein the target nucleotide sequence comprises all or a part of a nucleotide sequence located on a coding strand of a transcribed region of the target gene. Numbered embodiment 14 comprises the bacteriophage of any one of embodiments 1-13, wherein the target nucleotide sequence comprises at least a portion of an essential bacterial gene that is needed for survival of the target bacterium. Numbered embodiment 15 comprises the bacteriophage of embodiments 1-14, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. Numbered embodiment 16 comprises the bacteriophage of any one of embodiments 1-15, wherein the target nucleotide sequence is in a non-essential bacterial gene or genomic locus. Numbered embodiment 17 comprises the bacteriophage of any one of embodiments 1-16, wherein the first nucleic acid sequence is a first CRISPR array further comprising at least one repeat sequence. Numbered embodiment 18 comprises the bacteriophage of embodiments 1-17, wherein the at least one repeat sequence is operably linked to the first spacer sequence at either its 5′ end or its 3′ end. Numbered embodiment 19 comprises the bacteriophage of any one of embodiments 1-18, wherein the first nucleic acid is inserted into a non-essential bacteriophage gene or other genomic locus. Numbered embodiment 20 comprises the bacteriophage of embodiments 1-19, wherein the non-essential gene is gp49, gp75, hoc, gp0.7, gp4.3, gp4.5, gp4.7, gp0.6, gp0.65, gp0.7, gp4.3, or gp4.5. Numbered embodiment 21 comprises the bacteriophage of any one of embodiments 1-20, wherein the target bacterium is C. difficile. Numbered embodiment 22 comprises the bacteriophage of any one of embodiments 1-21, wherein the bacteriophage is ϕCD146 or ϕCD24-2. Numbered embodiment 23 comprises the bacteriophage of any one of embodiments 1-22, wherein the target bacterium is killed by the lytic activity of the bacteriophage, by the activity of a CRISPR-Cas system using the first spacer sequence or the crRNA transcribed therefrom, or both. Numbered embodiment 24 comprises the bacteriophage of embodiments 1-23, wherein the CRISPR-Cas system is endogenous to the target bacterium. Numbered embodiment 25 comprises the bacteriophage of embodiments 1-24, wherein the CRISPR-Cas system is exogenous to the target bacterium. Numbered embodiment 26 comprises the bacteriophage of any one of embodiments 1-25, wherein the CRISPR-Cas system is a Type I CRISPR-Cas system, a Type II CRISPR-Cas system, or a Type III CRISPR-Cas system. Numbered embodiment 27 comprises the bacteriophage of any one of embodiments 1-26, wherein the CRISPR-Cas system is a Type I CRISPR-Cas system. Numbered embodiment 28 comprises a temperate bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the 1247 cI repressor region. Numbered embodiment 29 comprises the temperate bacteriophage of embodiments 1-28, wherein the temperate bacteriophage infects multiple bacterial strains. Numbered embodiment 30 comprises the temperate bacteriophage of any one of embodiments 1-29, wherein the target nucleotide sequence comprises all or a part of a promoter sequence for the target gene. Numbered embodiment 31 comprises the temperate bacteriophage of any one of embodiments 1-30, wherein the target nucleotide sequence comprises all or a part of a nucleotide sequence located on a coding strand of a transcribed region of the target gene. Numbered embodiment 32 comprises the temperate bacteriophage of any one of embodiments 1-31, wherein the target nucleotide sequence comprises at least a portion of an essential bacterial gene that is needed for survival of the target bacterium. Numbered embodiment 33 comprises the temperate bacteriophage of embodiments 1-32, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. Numbered embodiment 34 comprises the temperate bacteriophage of any one of embodiments 1-33, wherein the target nucleotide sequence is in a non-essential bacterial gene or genomic locus. Numbered embodiment 35 comprises the temperate bacteriophage of any one of embodiments 1-34, wherein the first nucleic acid sequence is a first CRISPR array further comprising at least one repeat sequence. Numbered embodiment 36 comprises the temperate bacteriophage of embodiments 1-35, wherein the at least one repeat sequence is operably linked to the first spacer sequence at either its 5′ end or its 3′ end. Numbered embodiment 37 comprises the temperate bacteriophage of any one of embodiments 1-36, wherein the first nucleic acid is inserted into a non-essential bacteriophage gene or other genomic locus. Numbered embodiment 38 comprises the temperate bacteriophage of embodiments 1-37, wherein the non-essential bacteriophage gene is gp49, gp75, hoc, gp0.7, gp4.3, gp4.5, gp4.7, gp0.6, gp0.65, gp0.7, gp4.3, or gp4.5. Numbered embodiment 39 comprises the temperate bacteriophage of any one of embodiments 1-38, wherein the target bacterium is C. difficile. Numbered embodiment 40 comprises the temperate bacteriophage of any one of embodiments 1-39, wherein the temperate bacteriophage is ϕCD146 or ϕCD24-2. Numbered embodiment 41 comprises the temperate bacteriophage of any one of embodiments 1-40, wherein the target bacterium is killed by the lytic activity of the temperate bacteriophage, by the activity of a CRISPR-Cas system using the first spacer sequence or the crRNA transcribed therefrom, or both. Numbered embodiment 42 comprises the temperate bacteriophage of embodiments 1-41, wherein the CRISPR-Cas system is endogenous to the target bacterium. Numbered embodiment 43 comprises the temperate bacteriophage of embodiments 1-42, wherein the CRISPR-Cas system is exogenous to the target bacterium. Numbered embodiment 44 comprises the temperate bacteriophage of any one of embodiments 1-43, wherein the CRISPR-Cas system is a Type I CRISPR-Cas system, a Type II CRISPR-Cas system, or a Type III CRISPR-Cas system. Numbered embodiment 45 comprises the temperate bacteriophage of any one of embodiments 1-44, wherein the CRISPR-Cas system is a Type I CRISPR-Cas system. Numbered embodiment 46 comprises a pharmaceutical composition comprising: Numbered embodiment (a) a bacteriophage of any one of embodiments 1-27, or a temperate bacteriophage of any one of embodiments 28-45; and (b) a pharmaceutically acceptable excipient. Numbered embodiment 47 comprises the pharmaceutical composition of embodiment 46, wherein the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, or any combination thereof. Numbered embodiment 48 comprises a method for killing a target bacterium, the method comprising introducing into the target bacterium a temperate bacteriophage comprising a first nucleic acid sequence encoding a first spacer sequence or a crRNA transcribed therefrom, wherein the first spacer sequence is complementary to a target nucleotide sequence from a target gene in the target bacterium, provided that the bacteriophage is rendered lytic by a 1247 cI repressor region knockout, thereby killing the target bacterium. Numbered embodiment 49 comprises the method of embodiments 1-48, wherein the temperate bacteriophage infects multiple bacterial strains. Numbered embodiment 50 comprises the method of any one of embodiments 1-49, wherein the target nucleotide sequence comprises all or a part of a promoter sequence for the target gene. Numbered embodiment 51 comprises the method of any one of embodiments 1-50, wherein the target nucleotide sequence comprises all or a part of a nucleotide sequence located on a coding strand of a transcribed region of the target gene. Numbered embodiment 52 comprises the method of any one of embodiments 1-51, wherein the target nucleotide sequence comprises at least a portion of an essential bacterial gene that is needed for survival of the target bacterium. Numbered embodiment 53 comprises the method of embodiments 1-52, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK. Numbered embodiment 54 comprises the method of any one of embodiments 1-53, wherein the target nucleotide sequence is in a non-essential bacterial gene or genomic locus. Numbered embodiment 55 comprises the method of any one of embodiments 1-54, wherein the first nucleic acid sequence is a first CRISPR array further comprising at least one repeat sequence. Numbered embodiment 56 comprises the method of embodiments 1-55, wherein the at least one repeat sequence is operably linked to the first spacer sequence at either its 5′ end or its 3′ end. Numbered embodiment 57 comprises the method of any one of embodiments 1-56, wherein the first nucleic acid is inserted into a non-essential bacteriophage gene. Numbered embodiment 58 comprises the method of embodiments 1-57, wherein the non-essential bacteriophage gene is gp49, gp75, hoc, gp0.7, gp4.3, gp4.5, gp4.7, gp0.6, gp0.65, gp0.7, gp4.3, or gp4.5. Numbered embodiment 59 comprises the method of any one of embodiments 1-58, wherein the target bacterium is C. difficile. Numbered embodiment 60 comprises the method of any one of embodiments 1-59, wherein the temperate bacteriophage is ϕCD146 or ϕCD24-2. Numbered embodiment 61 comprises the method of any one of embodiments 1-60, wherein the target bacterium is killed by the lytic activity of the temperate bacteriophage, by the activity of a CRISPR-Cas system using the first spacer sequence or the crRNA transcribed therefrom, or both. Numbered embodiment 62 comprises the method of any one of embodiments 1-61, wherein the target bacterium is killed by the activity of the CRISPR-Cas system independently of the lytic activity of the temperate bacteriophage. Numbered embodiment 63 comprises the method of any one of embodiments 1-62, wherein activity of the CRISPR-Cas system supplements or enhances lytic activity of the temperate bacteriophage. Numbered embodiment 64 comprises the method of any one of embodiments 1-63, wherein lytic activity of the temperate bacteriophage and activity of the CRISPR-Cas system are synergistic. Numbered embodiment 65 comprises the method of any one of embodiments 1-64, wherein lytic activity of the temperate bacteriophage, activity of the CRISPR-Cas system, or both is modulated by a concentration of the temperate bacteriophage. Numbered embodiment 66 comprises the method of any one of embodiments 1-65, wherein the CRISPR-Cas system is endogenous to the target bacterium. Numbered embodiment 67 comprises the method of any one of embodiments 1-66, wherein the CRISPR-Cas system is exogenous to the target bacterium. Numbered embodiment 68 comprises the method of any one of embodiments 1-67, wherein the CRISPR-Cas system is a Type I CRISPR-Cas system, a Type II CRISPR-Cas system, or a Type III CRISPR-Cas system. Numbered embodiment 69 comprises the method of any one of embodiments 1-68, wherein the CRISPR-Cas system is a Type I CRISPR-Cas system. Numbered embodiment 70 comprises the method of any one of embodiments 1-69, wherein the temperate bacteriophage does not confer any new properties onto the target bacterium beyond cellular death caused by the lytic activity of the temperate bacteriophage, beyond the activity of the CRISPR-Cas array, or both. Numbered embodiment 71 comprises a method of treating a disease in an individual in need thereof, the method comprising administering the pharmaceutical composition of any one of embodiments 46-47. Numbered embodiment 72 comprises the method of embodiments 1-71, wherein the individual is a mammal. Numbered embodiment 73 comprises the method of any one of embodiments 1-72, wherein the disease is a bacterial infection. Numbered embodiment 74 comprises the method of embodiments 1-73, wherein a bacterium causing the bacterial infection is an Escherichia coli, Salmonella enterica, Bacillus subtilis, Clostridium acetobutylicum, Clostridium ljungdahlii, Clostridioides difficile, Clostridium bolteae, Acinetobacter baumannii, Mycobacterium tuberculosis, Mycobacterium abscessus, Mycobacterium intracellulare, Mycobacterium fortuitum, Mycobacterium chelonae, Mycobacterium avium, Mycobacterium gordonae, Myxococcus xanthus, Streptococcus pyogenes, cyanobacteria, Staphylococcus aureus, methicillin resistant Staphylococcus aureus, Streptococcus pneumoniae, carbapenem-resistant Enterobacteriaceae, extended spectrum beta-lactamase (ESBL)-producing Enterobacteriaceae, Staphylococcus epidermidis, Staphylococcus salivarius, Corynebacterium minutissimum, Corynebacterium pseudodiphtheriticum, Corynebacterium striatum, Corynebacterium group G1, Corynebacterium group G2, Streptococcus mitis, Streptococcus sanguinis, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces israelii., Porphyromonas gingivalis., Prevotella melaninogenicus, Helicobacter pylori, Helicobacter felis, Enterococcus faecalis, Enterococcus faecium, Enterococcus gallinarum, Bacteroides fragilis, Bacteroides thetaiotaomicron, Fusobacterium nucleatum, Ruminococcus gnavus, or Campylobacter jejuni or any combination thereof. Numbered embodiment 75 comprises the method of embodiments 1-74, wherein the bacterium is a drug resistant bacterium that is resistant to at least one antibiotic. Numbered embodiment 76 comprises the method of embodiments 1-75, wherein the bacterium is a multi-drug resistant bacterium that is resistant to at least one antibiotic. Numbered embodiment 77 comprises the method of any one of embodiments 1-76, wherein the at least one antibiotic comprises a cephalosporin, a fluoroquinolone, a carbapenem, a colistin, an aminoglycoside, vancomycin, streptomycin, or methicillin. Numbered embodiment 78 comprises the method of any one of embodiments 1-77, wherein the administering is intra-arterial, intravenous, intramuscular, oral, subcutaneous, inhalation, or any combination thereof.

### EXAMPLES

### Example 1. Bacterial Strains and Culture Conditions

All bacterial strains were stored at −80° C. in their respective medium supplemented with a final concentration of 15% glycerol (vol/vol) or as spore stocks. The C. difficile strains used herein were provided by Louis-Charles Fortier and Seth Walk. Strains were struck from freezer stocks onto brain heart infusion (BHI) agar plates (Teknova) and incubated at 37° C. in a Coy anaerobic chamber using 85% nitrogen, 5-10% hydrogen and 5% carbon dioxide. Strains were sub-cultured by inoculating BHI broth with a single colony and incubating at 37° C. BHI agar was supplemented with cycloserine (8 μg/mL), cefoxitin (25 μg/mL), and thiamphenicol (Tm) (15 μg/mL) to select for recombinant C. difficile. Escherichia coli strains were streaked onto LB agar plates (Teknova) and incubated at 37° C. E. coli strains were grown in LB broth, which was supplemented where necessary with carbenicillin (50 μg/mL), chloramphenicol (15 μg/mL), or erythromycin (200 μg/mL).

Table 1 shows the bacterial strains and plasmids used in the examples disclosed herein.

TABLE 1
Bacterial strains and plasmids

| Species | Strain | Purpose | Source |
|---|---|---|---|
| E. coli | DH5α | Cloning host | NEB |
| E. coli | SD46 | Conjugation donor | Theriot |
| C. difficile | CD19 | Amplification host; | Fortier |
| | | animal model target | |
| C. difficile | CD24 | ϕCD24-2 lysogen | Fortier |

| Plasmid | Purpose | Source |
|---|---|---|
| pMTL84151 | Cloning vector, | Chain Biotech |
| | Conjugation control | |
| pMTL82151 | Cloning vector | Chain Biotech |
| pMTL82251 | Cloning vector | Chain Biotech |
| pMTL84151::CRISPR | CRISPR validation | Locus |

### Example 2. DNA Isolation and Engineering

All kits and reagents were used according to the manufacturer's instructions. Plasmid and genomic DNA isolation was performed using the Zymo Research Plasmid Miniprep Kit and the Quick DNA Fungal/Bacterial Miniprep kit, respectively. Phage DNA purification was performed using standard procedures. Restriction enzymes, T4 DNA ligase, and DNA polymerases were from New England Biolabs. Routine PCR was performed using Taq DNA polymerase and high-fidelity amplifications were performed using Phusion DNA polymerase. All PCR products were visualized by gel electrophoresis using 0.8% agarose with Gel Red. Recombinant phages were created containing an expression cassette encoding a bacterial genome-targeting CRISPR RNA and/or genetic knockouts within putative lysogeny control modules in the phage genome.

### Example 3. Phage Morphology

Phages were prepared for TEM using a modification of the method described by Fortier and Moineau. Prior to observation, 1.5 mL of crude lysate was centrifuged for 1 hr at 4° C. and 24,000×g. A fraction of the supernatant (approximately 1.4 mL) was gently discarded, and 1 mL of ammonium acetate (0.1 M, pH 7.5) was added to the remaining lysate, which was then centrifuged as described above. This step was performed twice. Washed phage samples were visualized by negative-stain transmission electron microscopy. A glow-discharged formvar/carbon-coated 400 mesh copper grid (Ted Pella, Inc., Redding, Calif.) was floated on a 25-μL droplet of the sample suspension for five min, transferred quickly to two drops of deionized water followed by a droplet of 2% aqueous uranyl acetate stain for 30 sec. The grid was blotted with filter paper and air-dried. Samples were observed using a JEOL JEM-1230 transmission electron microscope operating at 80 kV (JEOL USA, Peabody, Mass.) and images were taken using a Gatan Orius SC1000 CCD camera with Gatan Microscopy Suite 3.0 software (Gatan, Inc., Pleasanton, Calif.).

### Example 4. Phage Handling Procedures

ϕCD24-2 in the prophage state was induced from C. difficile CD24 by UV irradiation (302 nm). Both the wild type ϕCD24-2 (wtPhage) and the CRISPR-enhanced phage (crPhage) were propagated by amplification on C. difficile CD19. An overnight culture of C. difficile CD19 was sub-cultured 1:100 into BHI broth and incubated at 37° C. to an OD₆₀₀ of 0.10. MgCl₂ and CaCl₂ were added to final concentrations of 10 mM and 1 mM, respectively. Bacteriophage was added at a multiplicity of infection (MOI) of 0.02 and cultures were incubated at 37° C. for 7-8 hours, until some clearance of the culture was observed. Amplification cultures were removed from the anaerobic chamber and centrifuged at 4000 g for 20 min. Supernatants were filtered through 0.45 μm filters. Phages were PEG-precipitated by adding 0.2 volumes of 20% PEG8000, 2.5 M NaCl solution (Teknova catalog #P4137) and incubating overnight at 4° C. The following day, phage suspensions were centrifuged for 10 min at 13,000 g at 4° C. Supernatants were decanted and pellets were resuspended in 5 mL BHI. Phage suspensions were centrifuged for 10 min at 13,000 g at 22° C. to remove residual PEG. Supernatants were transferred to fresh tubes and 10 mM MgCl₂ and 1 mM CaCl₂ were added. Lysates were stored at 4° C. until use. Phage titer was determined by the soft agar overlay method. Briefly, 800 μL of 2 M MgCl₂, 20 μL of 2 M CaCl₂, 500 μL of C. difficile CD19 at an OD₆₀₀ of 0.3-0.6, and 100 μL of phage at a range of dilutions were added to 3 mL of 0.375% BHI agar (Teknova). The mixture was poured onto a 1.5% BHI agar plate, allowed to solidify, and incubated at 37° C. anaerobically overnight. The following day, plaques were counted and the number of phages per ml was calculated.

### Example 5. In Vitro Phage Efficacy

Phages were diluted to a titer of 2.0×10⁸ PFU/mL in BHI+10 mM MgCl₂+1 mM CaCl₂. Overnight cultures of C. difficile were subcultured 1:100 into BHI and incubated at 37° C. to an OD₆₀₀ of 0.20. 10 mM MgCl₂ and 1 mM CaCl₂ were added to the bacterial culture, and culture was mixed 1:1 with phage or BHI+10 mM MgCl₂+1 mM CaCl₂. At 0, 2, 4, 6, and 22 hour, 10-fold serial dilutions were made in BHI down to 1:10⁶. A 5-4, volume of each dilution was spotted onto BHI agar and allowed to dry into the surface of the plate. Plates were incubated overnight at 37° C. The following day, the number of colonies in the densest countable spot was determined and used to calculate the number of cells per ml. This experiment was repeated with wtPhage and crPhage; wtPhage and wtPhage Δlys; and with wtPhage, crPhage, wtPhage Δlys, and crPhages Δlys.

### Example 6. Toxin Gene Expression In Vitro

Wild type C. difficile CD19 and CD19 lysogened by ϕCD24-2 were grown overnight in TY media, then subcultured into 10 ml fresh TY. Triplicate cultures for each strain were fixed at 24, 48, and 72 hour by adding 10 ml of cold 1:1 ethanol:acetone; these were immediately removed from the anaerobic chamber and stored at −80° C. overnight. Thawed samples were centrifuged at 3,000 g for 10 min at 4° C. Pellets were re-suspended in 1 ml of cold sterile water supplemented 1:100 with 2-mercaptoethanol, then centrifuged at 5,000 rpm for 5 min at 4° C. Supernatants were removed, and the RNA was extracted using the PureLink RNA Mini Kit (Thermo Fisher, cat. no. 12183018A) following the manufacturer's protocol. Contaminating DNA was removed with Turbo DNase (Thermo Fisher cat. no. AM2238), and cDNA was synthesized by use of Moloney Murine Leukemia Virus Reverse Transcriptase. The cDNA was then diluted 1:3 in sterile water. Quantitative real-time PCR was performed using the SsoAdvanced SYBR Green Supermix with primers specific to the genes in Table 2.

TABLE 2

| Gene | Forward primer | Reverse primer |
|---|---|---|
| rpoC | TGGCAGTCCATGTACCTTTATC | GGTGAACCATCTTTAGG |
| | | AGCA |
| tcdA | ACTAGACGAACATGACCCATTAC | GCTACCGTTGCAGCTAT |
| | | AGATAA |
| tcdB | GGCAGCTGCTTCTGACATATTA | GGTCTGGTTGTATTCCT |
| | | GGTAAC |

PCR amplifications were performed in technical duplicates. The copy numbers were calculated using a standard curve and relative copy numbers were obtained by normalizing tcdA and tcdB copy numbers to that of rpoC.

### Example 7. C. difficile Spore Preparation

Spores of C. difficile strain CD19 were prepared. Briefly, 2 mL of an overnight culture of CD19 in Columbia broth was added to 40 mL of Clospore media and incubated at 37° C. for 7 days, after which time the spores were centrifuged and washed 5 times in cold sterile water. Alternatively, 500 μL of an early log phase growth culture of C. difficile was spread onto a 70% SMC-30% BHI agar plate and incubated at 37° C. for 3-4 days. Growth was then scraped off of the agar plate and resuspended in 10 mL of sterile PBS. 10 mL of 96% ethanol was added, and the mixture was vortexed and allowed to sit on the bench top for one hour. The spore mix was then centrifuged at 3,000 rpm for 10 min. The pellet was suspended in 10 mL sterile PBS and centrifuged again. The pellet was then suspended in 1 mL sterile PBS and heated to 65° C. for 20 min. Spores prepared by either method were enumerated at the time of their preparation and prior to preparation of the inoculum via serial dilution in the anaerobic chamber and plating onto BHIS supplemented with 0.1% taurocholate. Spore inocula were also enumerated immediately prior to in vivo challenge.

### Example 8. Antibiotic Administration and Infection with C. difficile

Animals and housing: Male and female C57BL/6 mice (aged 5 weeks old) were purchased from Jackson Labs (Bar Harbor, Me.) for use in infection experiments. The food, bedding, and water were autoclaved, and all cage changes were performed in a laminar flow hood. The mice were subjected to a 12 hr light and 12 hr dark cycle. Animal experiments were conducted in the Laboratory Animal Facilities located on the North Carolina State University CVM campus. The animal facilities are equipped with a full time animal care staff coordinated by the Laboratory Animal Resources (LAR) division at NCSU. The NCSU CVM is accredited by the Association for the Assessment and Accreditation of Laboratory Animal Care International (AAALAC). Trained animal handlers in the facility fed and assessed the status of animals several times per day. Those assessed as moribund were humanely euthanized by CO₂ asphyxiation.

The mice (n=80 over three experiments, male and female) were administered 0.5 mg/mL of cefoperazone (dissolved in Gibco distilled water, catalog no. 15230147) in their drinking water for five days to render them susceptible to C. difficile colonization. All mice were then given distilled water to drink for two days, after which they were orally gavaged with 10⁵ spores of C. difficile strain CD19 in 25 μL. Animals were monitored for clinical signs of disease (weight loss, inappetence, wet stool, hunched posture, ruffled fur), and animals were humanely sacrificed via CO₂ asphyxiation if they met the clinical end point of loss of 20% of their initial body weight. Fecal pellets were collected daily, weighed, passed into the anaerobic chamber, and diluted 1:10 w/v in sterile anaerobic PBS (Gibco, catalog no. 10010023). The diluted pellets were serially diluted and plated onto the C. difficile selective media Cefoxitin d-Cycloserine Fructose agar (CCFA) to enumerate vegetative cells, and Taurocholate Cefoxitin d-Cycloserine Fructose agar (TCCFA) to enumerate the total C. difficile burden (vegetative cells and spores). Necropsy was performed on days 2 and 4 post challenge. Cecal content was harvested for enumeration of C. difficile on CCFA and TCCFA. Cecal and colon tissue was harvested for histopathology analysis.

### Example 9. Treatment with Phage

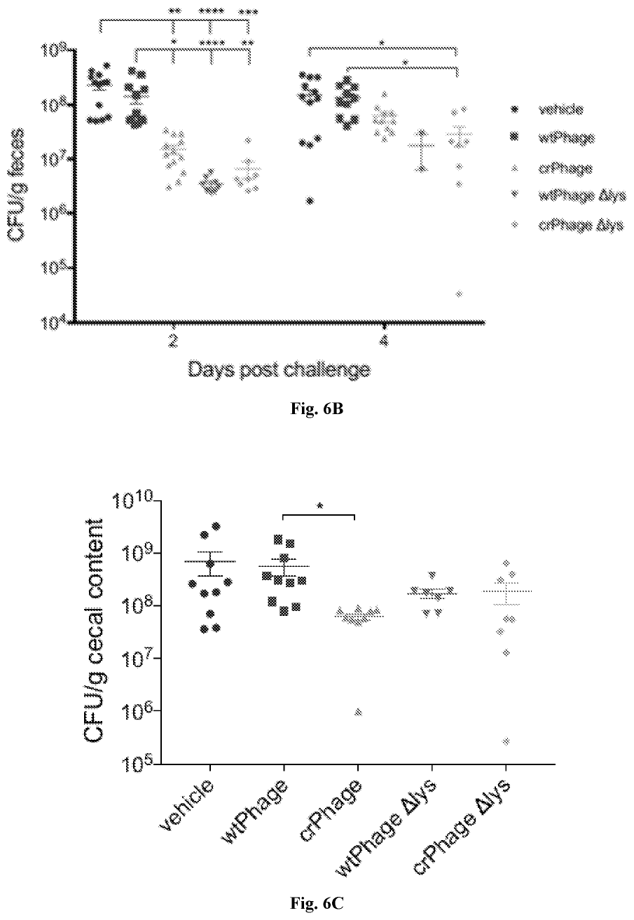

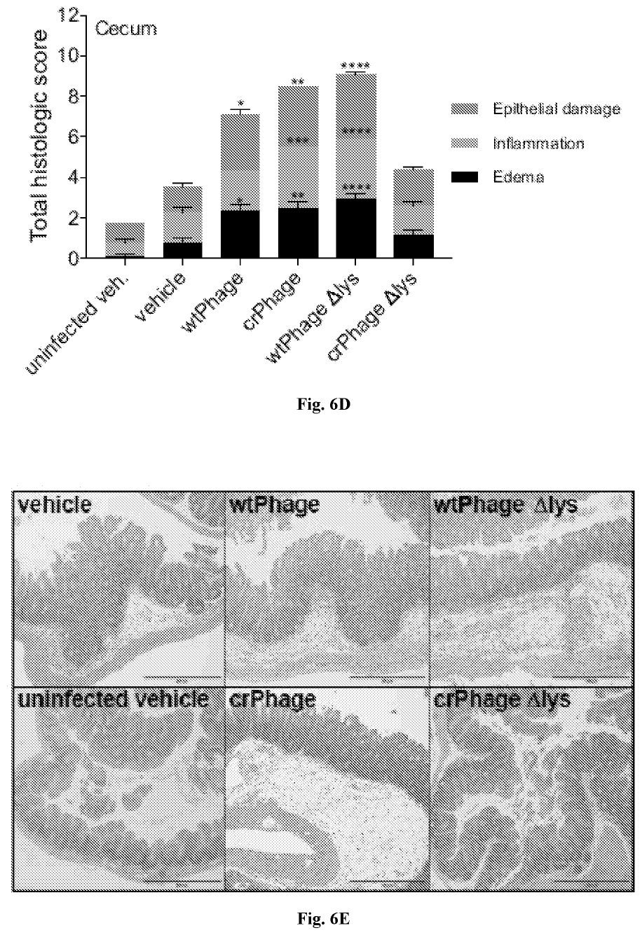

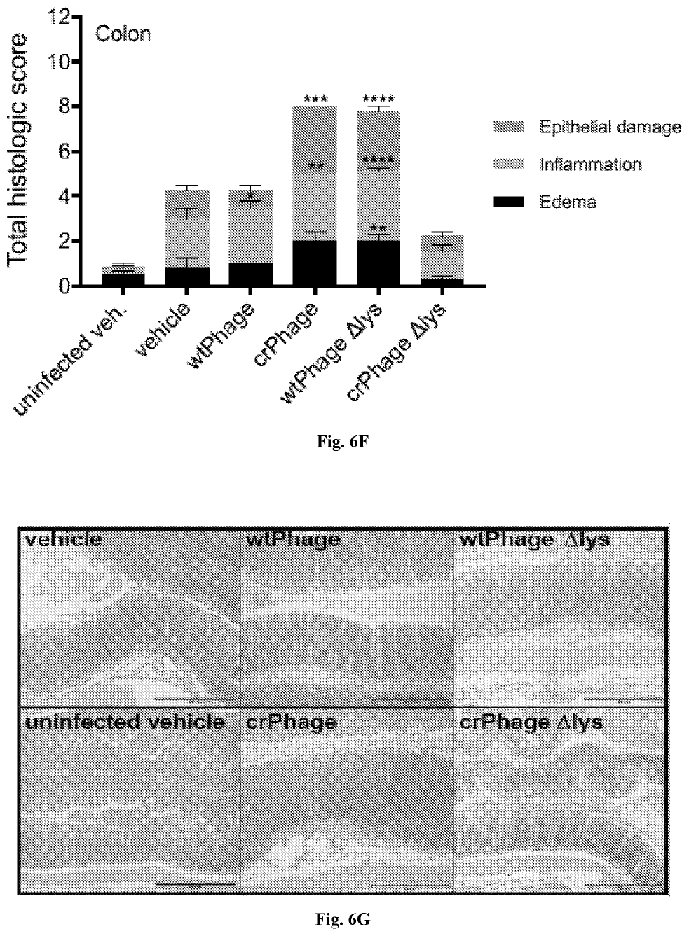

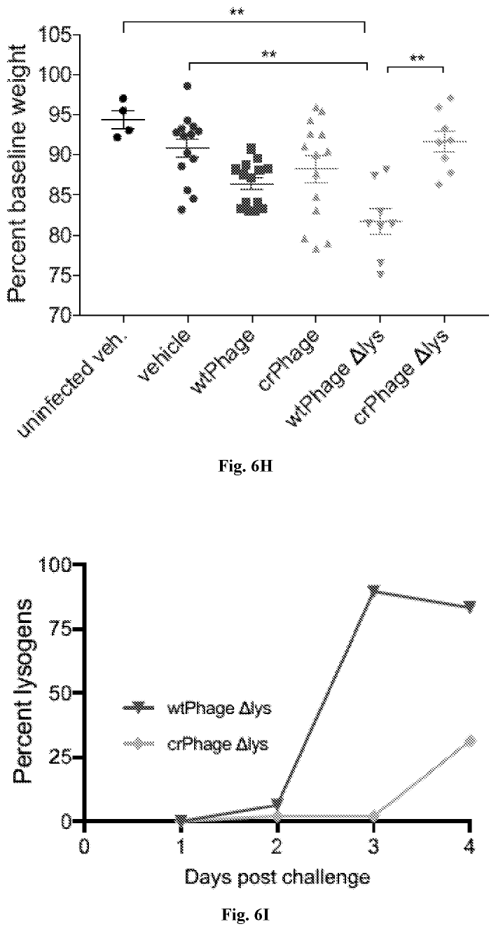

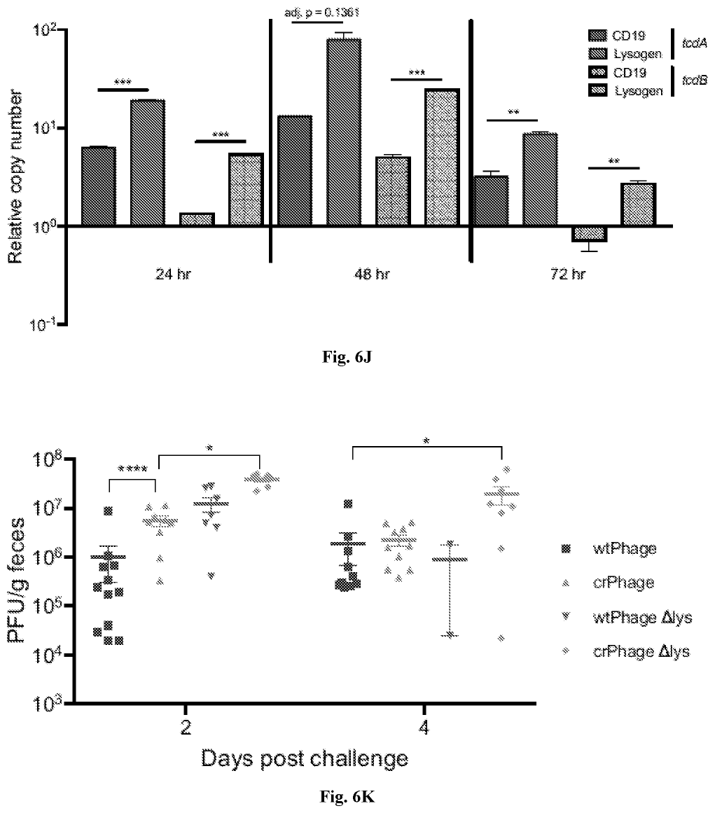

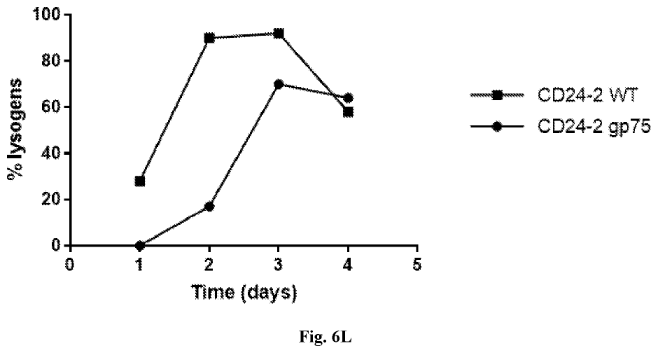

Approximately 4 hr after challenge with C. difficile spores, mice were orally gavaged with 100 μL of 6% w/v NaHCO₃ solution to neutralize stomach acid. After approximately 30 min, this was followed by one of five treatments in 100 μL: vehicle, which is BHI+10 mM MgCl₂+1 mM CaCl₂ (CD19, n=20); wtPhage (n=20); recombinant crPhage (n=20); wtPhage lysogeny mutant (wtPhage Δlys, n=8); or crPhage lysogeny mutant (n=8, crPhage Δlys). One group was given cefoperazone and vehicle, but was not challenged with C. difficile spores (n=4). This group served as the control for the effects of the vehicle on gut tissue in the histopathological analysis. The treatment gavages were repeated twice daily, approximately 8-9 hr apart, for the duration of the experiment (FIG. 6A).

### Example 10. Screening for Lysogens

Resuspended fecal pellets were plated on BHI agar supplemented with cycloserine and cefoxitin. Individual colonies were re-streaked onto BHI agar twice to isolate bacteria away from phage particles. Colonies were PCR screened using primers to detect the presence of a phage lysogen. Colonies positive for prophage presence were further screened using primers specific to wild type or CRISPR engineered phage. For titration of phage in fecal samples, resuspended fecal pellets were centrifuged for 10 min at 4000 g. Supernatants were filtered through 0.45 μm spin filters and used in soft agar overlays.

### Example 11. Histopathological Examination of the Mouse Cecum and Colon

At the time of necropsy, the cecum and colon were prepared for histology by placing the intact tissue into histology cassettes and stored in 10% buffered formalin for 48 hr, then transferred to 70% ethyl alcohol for long term storage. Tissue cassettes were further processed and paraffin embedded, then sectioned. Haematoxylin and eosin stained slides were prepared for histopathological examination (University of North Carolina Animal Histopathology & Lab Medicine core). Histological sections were coded, randomized, and scored in a blinded manner by a board-certified veterinary pathologist (SM). Edema, inflammation (cellular infiltration), and epithelial damage for the cecum and colon were each scored 0-4 based on a numerical scoring scheme.

Edema scores were as follows: 0, no edema; 1, mild edema with minimal (2×) multifocal submucosal expansion or a single focus of moderate (2-3×) sub-mucosal expansion; 2, moderate edema with moderate (2-3×) multifocal sub-mucosal expansion; 3, severe edema with severe (3×) multifocal sub-mucosal expansion; 4, same as score 3 with diffuse sub-mucosal expansion.

Cellular infiltration scores were as follows: 0, no inflammation; 1, minimal multifocal neutrophilic inflammation of scattered cells that do not form clusters; 2, moderate multifocal neutrophilic inflammation (greater submucosal involvement); 3, severe multifocal to coalescing neutrophilic inflammation (greater submucosal±mural involvement); 4, same as score 3 with abscesses or extensive mural involvement.

Epithelial damage was scored as follows: 0, no epithelial changes; 1, minimal multifocal superficial epithelial damage (vacuolation, apoptotic figures, villus tip attenuation/necrosis); 2, moderate multifocal superficial epithelial damage (vacuolation, apoptotic figures, villus tip attenuation/necrosis); 3, severe multifocal epithelial damage (same as above)+/−pseudomembrane (intraluminal neutrophils, sloughed epithelium in a fibrinous matrix); 4, same as score 3 with significant pseudomembrane or epithelial ulceration (focal complete loss of epithelium).

Photomicrographs were captured on an Olympus BX43 light microscope with a DP27 camera using cellSens Dimension software.

### Example 12. Statistical Methods

Statistical tests were performed using Prism version 7.0b for Mac OS X (GraphPad Software, La Jolla Calif. USA). Statistical significance was set at a p value of <0.05 for all analyses (*p<0.05, **p<0.01, ***p<0.001, ****p<0.0001). For the in vivo experiments, a Kruskal-Wallis One-Way ANOVA followed by Dunn's multiple comparisons post hoc test was used to calculate significance between treatment groups. A Geisser-Greenhouse Two-Way ANOVA with Sidak's multiple comparisons post hoc test was used to calculate significance between in vitro toxin expression data. A Student's t-test corrected for multiple comparisons using the Holm-Sidak method was used to calculate significance between groups in conjugation efficiency, and Mann-Whitney two-tailed t-test to evaluate histologic scores in CD19 infection.

### Example 13. Type I-B CRISPR Cas System in C. difficile as an Antimicrobial Via Phage Delivery of a Genome-Targeting CRISPR Array

The C. difficile genome-targeting CRISPR was constructed using the native leader and consensus CRISPR repeat from the highly expressed endogenous CR11 array in C. difficile 630. 236 nucleotide of the leader sequence was selected, which drives expression of the CRISPR array from canonical 670 and RNA polymerase recognition motifs. The repeat sequence was generated by deriving the 29 nucleotide consensus of 15 repeats from the C. difficile 630 CR11 array. The optimal length of the spacer sequence was defined by the length distribution of all spacers in ˜220 queried genomes, which was determined to be 37 nucleotides; thus the spacer sequence was constrained to 37 nucleotides downstream of the consensus PAM sequence 5′-CCW-3′ and was selected for its high conservation across C. difficile strains. The CRISPR targeting sequence is shown below with the leader sequence (underlined), the repeat sequences (bolded) and the spacer sequence targeting ribonuclease Y (italicized):

SEQ ID NO: 1
GTGCTTTTAAATTTACAAAGTATTCCATTTTAATTTTATAGTTTAGATT
TTATGATATAATAAAAATATAGAAGTTTTGCAGTGTGCGATATTTGTTA
CAAAGTAGGGCTTAATACTTGAAATCTAAGATGTTGAGGGTGCGTGATA
AGTGTTATCAATTGCACTATTGCCCGCTCACTGCAATTTTAAGAGTATT
GTATATATGTAGGTATTGGAAATGCTAAGTTTATTTTGGGGTTTTAGAT
TAACTATATGGAATGTAAATGGTCTAGCAGCTGATATTGCATCTGCTGC
TGTAACTAGTTTTAGATTAACTATATGGAATGTAAAT

The CRISPR expression construct was then validated by cloning into pMTL84151 and subsequent conjugation into model C. difficile strains R20291 and 630. Successful genome-targeting by the CRISPR was defined by the reduction in the number of viable transconjugants of pMTL84151: CRISPR compared to the pMTL84151 vector control.

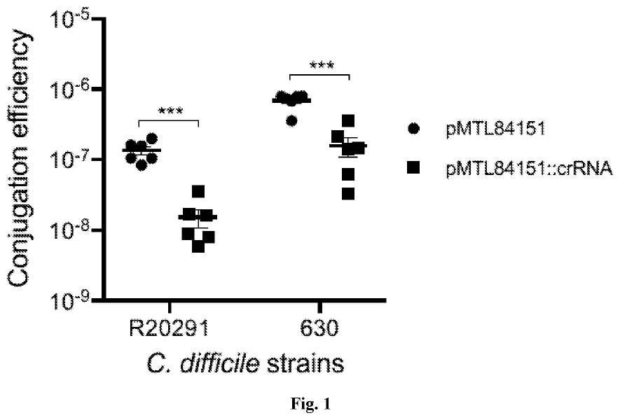

The CRISPR plasmid exhibited an approximately one-log reduction in conjugation efficiency (FIG. 1). The genome-targeting CRISPR caused a significant loss in viable transconjugants, implying lethal DNA degradation caused by the endogenous Type I-B system in C. difficile, and validating its use for improving the efficacy of bacteriophage-mediated bacterial suppression.

### Example 14. Phage-Mediated Delivery of a Genome-Targeting CRISPR Enhances Wildtype Phage Activity to More Efficiently Kill a Target Population of C. difficile

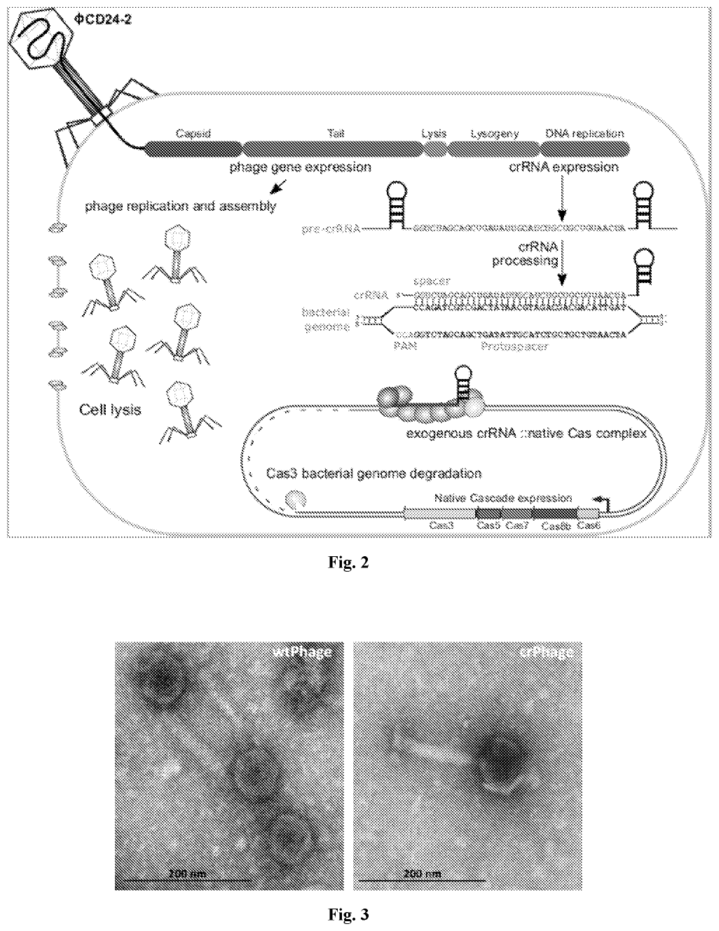

Given the efficacy of the genome-targeting CRISPR, the leader-driven repeat-spacer-repeat construct was moved onto the genome of the C. difficile bacteriophage ϕCD24-2 (CRISPR-enhanced phage and crPhage are used interchangeably) (FIG. 2). To determine whether genetic modification of the phage would affect its ultrastructural integrity or its stability, the phage morphology of wild-type ϕCD24-2 (wtPhage) was compared with that of the crPhage by negatively stained TEM (FIG. 3). Both the WT and crPhage displayed typical Myoviridae morphology. In terms of host range, wtPhage was capable of infecting 10/87 strains from a clinically relevant strain panel as determined by the spot plating assay. The crPhage infected the same 10/87 strains as the wtPhage, indicating that insertion of the CRISPR into the phage genome did not affect the morphology or host range. The titers achieved were assessed by routine amplification and storage stability of the crPhage was compared to that of the wtPhage and no differences were found between them over the course of four weeks.

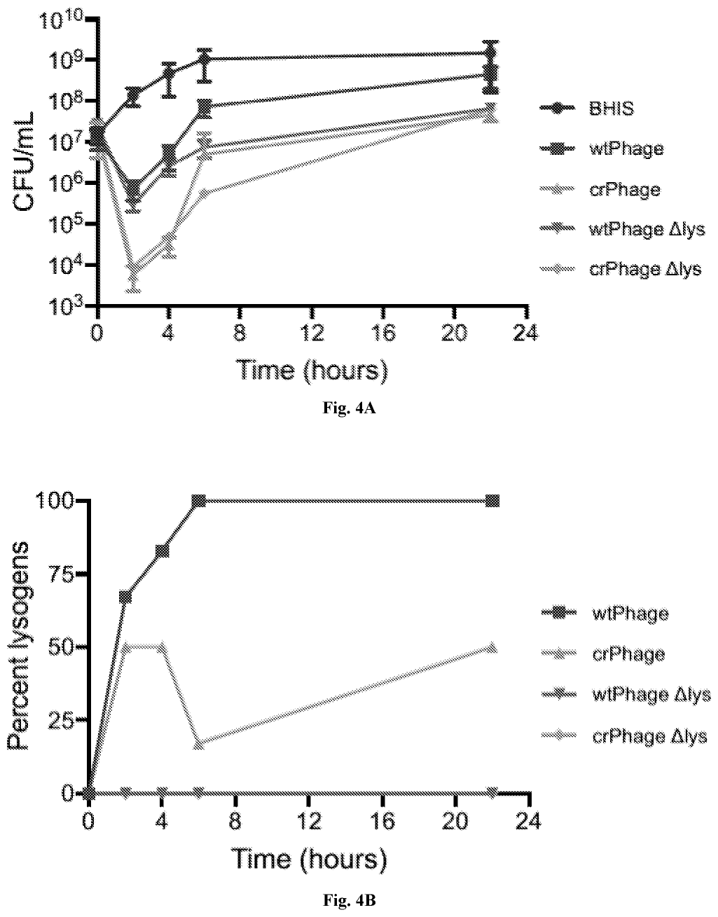

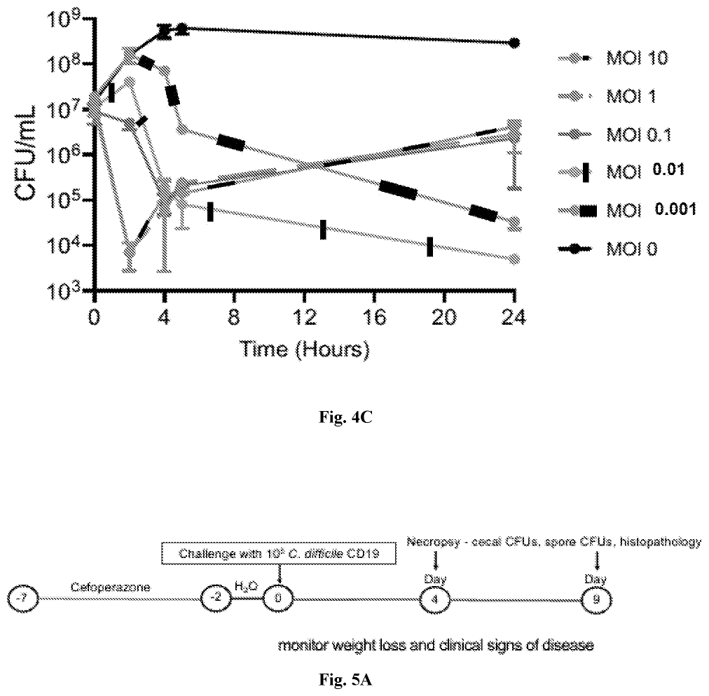

Next, in vitro colony forming unit (CFU) reduction experiments were performed against C. difficile strain CD19, which is a host highly susceptible to ϕCD24-2 infection. The cultures treated with wtPhage exhibited an approximately one-log reduction in CFUs after two hours of incubation, and quickly rebounded thereafter. In contrast, the crPhage both increased the depth of maximum CFU reduction after two hours of infection (approximately three logs), and decreased the recovery of the culture over 24 hours (FIG. 4A). The multiplicity of infection (MOI) used for treatment also played a role in the observed depth of killing and delayed rebound: MOI ≥1 favored rapid bacterial killing, but also facilitated rebound of the culture, whereas MOI ≥0.01 produced a higher depth of kill throughout the duration of the experiment (FIG. 4C). These CFU rebound data are consistent with the occurrence of bacteriophage insensitive mutants, which may be selected for at high initial MOIs. Specifically, lysogens are insensitive to reinfection, allowing them to grow to dominate the population despite the presence of active phages (FIG. 4A-FIG. 4B).

### Example 15. Treatment with the crPhage and the Effect on the Outcome of Disease in a Mouse Model of C. difficile Infection (CDI) in In Vivo

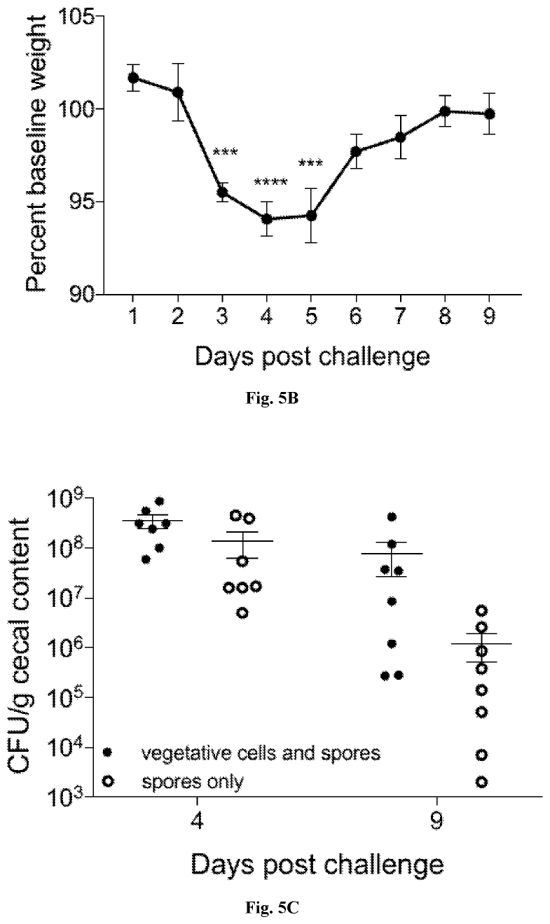

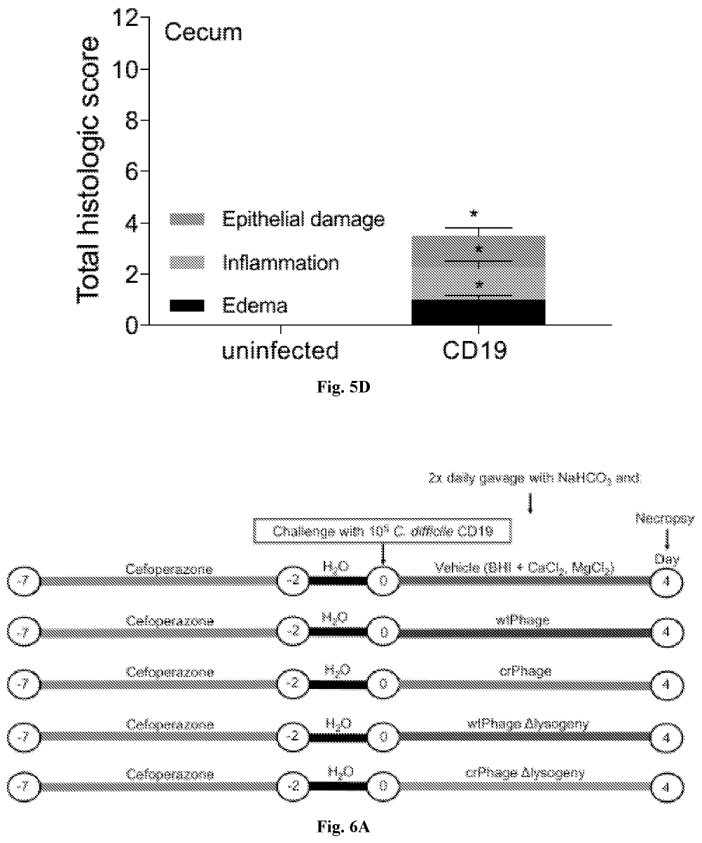

First, the kinetics of infection with C. difficile strain CD19 were determined. Mice were given the antibiotic cefoperazone in their drinking water for five days to make them susceptible to CDI, and then were challenged with C. difficile CD19 spores on day 0 (FIG. 5A). At four days post challenge, significant weight loss, high cecal burdens of C. difficile CD19, and significant histopathological changes to the cecum including edema, inflammation, and epithelial damage were observed (FIG. 5B-FIG. 5D).

Four days post challenge was selected as the endpoint for the experimental phage therapy model. As aforementioned, mice were given cefoperazone in their water, and four hours after spore challenge, the mice were given via oral gavage 100 μl of 6% NaHCO₃w/v in water to increase the pH of the stomach and protect administered phages from degradation during transit through the stomach. The mice then received one of three treatments via oral gavage: vehicle, wtPhage, or crPhage (FIG. 6A). Mice treated with the crPhage had a significantly reduced C. difficile burden, with an approximately 10-fold reduction in vegetative C. difficile CFUs in their feces two days post challenge, relative to mice given either vehicle alone or the wtPhage (p=0.0013 and p=0.0232) (FIG. 6B). In contrast, mice treated with the wtPhage had vegetative cell CFUs in their feces similar to mice treated with vehicle, suggesting that the wtPhage is not as virulent in vivo. By day 4, vegetative cell CFUs rebounded in mice treated with the crPhage, though the CFUs were still lower than those from vehicle- and wtPhage-treated mice. The cecal CFUs at the time of necropsy on day 4 also showed a significant reduction in C. difficile vegetative cell burden in the crPhage treated group compared to mice treated with wtPhage (p=0.0175, FIG. 6C). In some instances, the rebound in CFUs at day 4 is due to lysogeny, given the in vitro observations.

Six individual C. difficile colonies were PCR-screened from the feces of each mouse at two and four days post challenge. On day 2, it was found that 71% of the colonies screened from mice treated with wtPhage-contained lysogens, demonstrating efficient infection of the total C. difficile population in vivo, as depicted in FIG. 6L. By contrast, none of the colonies from day 2 mice treated with the crPhage contained lysogens. By day 4, all of the colonies screened from mice treated with wtPhage contained lysogens. Likewise, all colonies obtained from the day 4 feces of crPhage-treated mice were lysogens. The lysogens were confirmed by PCR to have the correct identity—that is, lysogens from groups treated with wtPhage contained the wild-type phage, and lysogens from groups treated with crPhage were recombinant. However, upon sequencing 35 crPhage lysogens from day 4 (representing all detected lysogens over two experiments), it was found that 30 of them had lost the spacer and one repeat from the CRISPR region. Four failed to produce good sequence data, and one maintained the spacer and both repeats. This observation indicates that, in some instances, lysogens with an intact genome-targeting CRISPR are unstable and select for excision of the CRISPR spacer, given that excision of spacers by repeat-mediated recombination is a primary form of escape from genome- and plasmid targeting.

### Example 16. Deletion of Lysogeny Modules of the Bacteriophage

Given the pervasive lysogeny by day 4, mutant phages lacking key lysogeny genes in the wtPhage and crPhage were constructed. CFU reduction assays were performed, and no lysogens were detected over the course of 22 hours from cultures treated with either phage in vitro (FIG. 4B). The in vivo mouse model of CDI was repeated, and mice were given either phage as treatment. Mice treated with the wtPhage Δlys had nearly two logs reduction in their fecal C. difficile burdens at two days post challenge relative to the mice given vehicle or wtPhage alone (p<0.0001 for both, FIG. 6B). By day 2, mice given the crPhage Δlys had a nearly four-fold reduction in fecal CFUs relative to mice treated with crPhage alone, and nearly a two-log reduction compared to vehicle-treated mice (FIG. 6B). By day 4, fecal CFUs had increased in all treatment groups, though the mice given crPhage Δlys still had significantly lower fecal CFUs than the mice given vehicle or those treated with the wtPhage (p=0.0472 and p=0.0125, FIG. 6B). The C. difficile burdens in the day 4 cecal content from mice treated with either wtPhage Δlys or crPhage Δlys were not significantly different from mice given vehicle, or from mice given the parent phage treatment (FIG. 6C). Significant histopathological changes were detected in the ceca (FIG. 6D-FIG. 6E) and colons (FIG. 6F-FIG. 6G) from mice treated with the crPhage and wtPhage Δlys; however, the cecal and colonic tissue, as well as weight loss from mice treated with the crPhage Δlys were not significantly different from that of uninfected, vehicle-treated control mice (FIG. 6D-FIG. 611), suggesting that the activity of this modified recombinant phage protected the host from the tissue damage that is associated with CDI.

Lysogens were detected in the feces of mice treated with each, albeit to a lower frequency with the latter phage (FIG. 6I). PCR screening confirmed that they were not the result of contamination with wtPhage or crPhage. The crPhage Δlys lysogens maintained the spacer and both repeats. The CD19 lysogen exhibited significantly increased expression of tcdA and tcdB over time in vitro (FIG. 6J), indicating that lysogeny, in some instances, affects the bacterial physiology of C. difficile by way of increased toxin gene expression. The increased toxin gene expression, in some instances, caused increased tissue pathology.

### Example 17. Engineering and Validation of cI-Knockout C. difficile Bacteriophage

Engineering: Plasmids containing homology arms flanking different portions of the lysogeny region in ϕCD24-2 and a counter selective crRNA targeting the lysogeny region were designed in silico and synthesized by BioBasic. The plasmid was transformed into E. coli and conjugated into C. difficile strain CD19. ϕCD24-2 was amplified on CD19 carrying the engineering plasmid. The resulting phage population was PCR screened for the presence of engineered phages. If the bulk PCR was positive, the lysate was plagued and individual plaques were screened for the appropriate lysogeny region knockout. Pure engineered phage was amplified to high titer for use in validation studies.

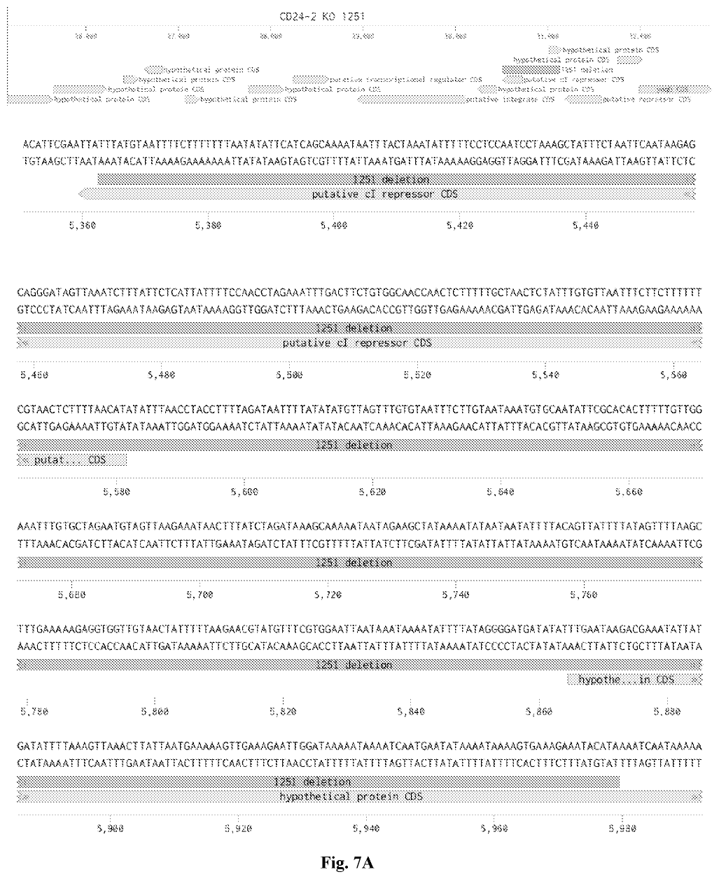

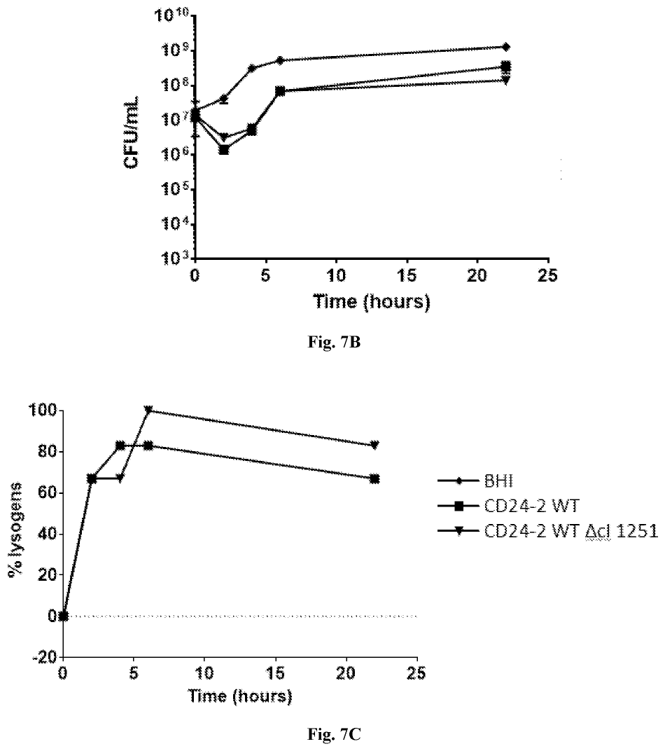

Validation: CD19 culture at mid-log phase was treated with WT or ΔcI CD24-2. The number of surviving cells was counted at various time points after treatment. Surviving cells were screened for the presence of lysogenized CD24-2 (FIG. 7-10).

The “1251 deletion” is illustrated in FIG. 7A. The 1251 phage variant did not affect phage kill, as depicted in FIG. 7B. The 1251 phage variant also did not affect lysogeny formation rate, as indicated in FIG. 7C.

The “1224 deletion” is illustrated in FIG. 8B. This engineered variant did not improve phage kill, as depicted in FIG. 8B. However, this variant did decrease lysogeny formation rate, as depicted in FIG. 8C.

The “1249 deletion” is illustrated in FIG. 9A. This engineered variant (“1249 variant” also referred to herein as “WT ΔcI”) significantly improved phage kill, as depicted in FIG. 9B. Further, the 1249 variant also significantly slows lysogeny formation rate, as depicted in FIG. 9C. The 1249 variant was combined with CRISPR RNA. As depicted in FIG. 9D, combining the 1249 variant with CRISPR RNA (gp75x1249) prevented lysogeny from occurring throughout the time course of the experiment.

The “1247 deletion” is illustrated in FIG. 10A. This engineered variant (“1247 variant”) enhanced phage kill, as depicted in FIG. 10B. Further, the 1249 variant prevented lysogeny from occurring, as depicted in FIG. 10C. Combining the 1249 variant with the genome targeting CRISPR RNA the gp75 gene produced similar levels of phage kill and did not affect the number of lysogens, as depicted in FIG. 10D-10E.

### Example 18: Lysogeny Removal in p1473

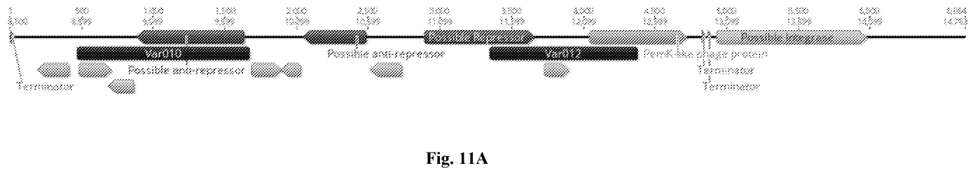

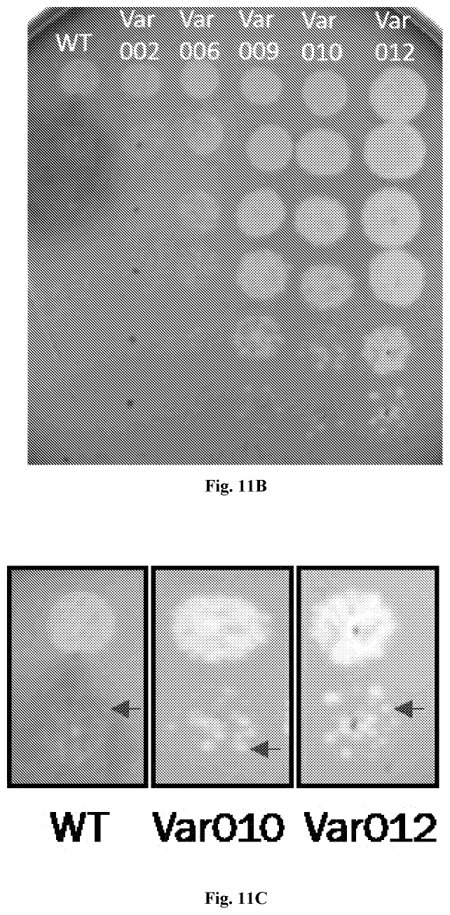

Deletion of the lysogeny region was tested in phage p1473. Open reading frames encoding putative repressor and anti-repressor proteins and an open reading frame encoding a putative integrase is shown in FIG. 11A. The first recombinant phage contains a genetic deletion that was introduced into the predicted lysogeny module region of the bacteriophage genome in the region including a putative anti-repressor and two clones were isolated and designated Var009 and Var010. A second recombinant phage contains a genetic deletion that was introduced into the predicted lysogeny module region of the bacteriophage genome in the region including a putative repressor and a single clone was isolated and designated Var012. Two additional control variants were generated with different deletions outside of the lysogeny region as controls (not shown), isolated and designated Var002 and Var006, respectively.

Wildtype p1473 (WT) and several variants were plated on a lawn of Staphylococcus aureus using the double agar overlay method The results are depicted in FIG. 11B. The WT phage and variants Var002 and Var006 produced small hazy plaques, while variants Var009, Var010 and Var012 produced larger clearer plaques. Variants Var002 and Var006 contain mutations outside of the lysogeny region. The variants with mutants outside the lysogeny region do not show changes in plaque morphology indicating that these phage variants remain temperate. Var009, Var010 and Var012, with mutations in the lysogeny zone show clearer plaques and higher efficiency of plaquing on the strain shown. These data indicate that Var010 and Var012 were successfully converted to lysogenic to lytic phenotype, as clear plaque morphology is a hallmark of lytic bacteriophages in S. aureus. The ability of Var010 and Var012 to plaque at higher efficiency (i.e. lower down the plate, when the phage is more dilute) may indicate that the modified phage is no longer susceptible to interference from the endogenous prophage present in the bacterial strain.

FIG. 11C depicts a close up image showing the larger plaque morphology for wildtype p14′73, Var010, and Var012. The arrows point to individual plaques. Var010 and Var012 plaques display a clearer morphology compared to wild-type. Additionally, the zone of clearing with too many plaques to count is clearer in Var010 and Var012 than the WT phage.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic.

2. The bacteriophage of claim 1, wherein the bacteriophage is derived from a temperate bacteriophage.

3. The bacteriophage of any one of claims 1-2, wherein the bacteriophage is rendered lytic by removal, replacement, or inactivation of a lysogenic gene.

4. The bacteriophage of any one of claims 1-3, wherein the bacteriophage is rendered lytic by removal of a 1247 cI repressor region.

5. The bacteriophage of any one of claims 1-3, wherein the bacteriophage is rendered lytic by the removal of a 1249 cI repressor region.

6. The bacteriophage of any one of claims 1-3, wherein the bacteriophage is rendered lytic by the removal of a 1224 cI repressor region.

7. The bacteriophage of any one of claims 1-3, wherein the bacteriophage is rendered lytic by the removal of a regulatory element of a lysogeny gene.

8. The bacteriophage of any one of claims 1-3, wherein the bacteriophage is rendered lytic by the removal, alteration or replacement of a promoter of a lysogeny gene.

9. The bacteriophage of any one of claims 1-3, wherein the bacteriophage is rendered lytic by the removal of a functional element of a lysogeny gene.

10. The bacteriophage of any one of claims 1-2, wherein the bacteriophage is rendered lytic via a second CRISPR array comprising a second spacer directed to a lysogenic gene.

11. The bacteriophage of any one of claims 1-10, wherein the bacteriophage infects multiple bacterial strains.

12. The bacteriophage of any one of claims 1-11, wherein the target nucleotide sequence comprises all or a part of a promoter sequence for the target gene.

13. The bacteriophage of any one of claims 1-11, wherein the target nucleotide sequence comprises all or a part of a nucleotide sequence located on a coding strand of a transcribed region of the target gene.

14. The bacteriophage of any one of claims 1-11, wherein the target nucleotide sequence comprises at least a portion of an essential bacterial gene that is needed for survival of the target bacterium.

15. The bacteriophage of claim 14, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK.

16. The bacteriophage of any one of claims 1-11, wherein the target nucleotide sequence is in a non-essential bacterial gene or genomic locus.

17. The bacteriophage of any one of claims 1-16, wherein the first nucleic acid sequence is a first CRISPR array further comprising at least one repeat sequence.

18. The bacteriophage of claim 17, wherein the at least one repeat sequence is operably linked to the first spacer sequence at either its 5′ end or its 3′ end.

19. The bacteriophage of any one of claims 1-18, wherein the first nucleic acid is inserted into a non-essential bacteriophage gene or other genomic locus.

20. The bacteriophage of claim 19, wherein the non-essential gene is gp49, gp75, hoc, gp0.7, gp4.3, gp4.5, gp4.7, gp0.6, gp0.65, gp0.7, gp4.3, or gp4.5.

21. The bacteriophage of any one of claims 1-20, wherein the target bacterium is C. difficile.

22. The bacteriophage of any one of claims 1-21, wherein the bacteriophage is ϕCD146 or ϕCD24-2.

23. The bacteriophage of any one of claims 1-22, wherein the target bacterium is killed by the lytic activity of the bacteriophage, by the activity of a CRISPR-Cas system using the first spacer sequence or the crRNA transcribed therefrom, or both.

24. The bacteriophage of claim 23, wherein the CRISPR-Cas system is endogenous to the target bacterium.

25. The bacteriophage of claim 23, wherein the CRISPR-Cas system is exogenous to the target bacterium.

26. The bacteriophage of any one of claims 23-25, wherein the CRISPR-Cas system is a Type I CRISPR-Cas system, a Type II CRISPR-Cas system, or a Type III CRISPR-Cas system.

27. The bacteriophage of any one of claims 23-26, wherein the CRISPR-Cas system is a Type I CRISPR-Cas system.

28. A temperate bacteriophage comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the 1247 cI repressor region.

29. The temperate bacteriophage of claim 28, wherein the temperate bacteriophage infects multiple bacterial strains.

30. The temperate bacteriophage of any one of claims 28-29, wherein the target nucleotide sequence comprises all or a part of a promoter sequence for the target gene.

31. The temperate bacteriophage of any one of claims 28-29, wherein the target nucleotide sequence comprises all or a part of a nucleotide sequence located on a coding strand of a transcribed region of the target gene.

32. The temperate bacteriophage of any one of claims 28-29, wherein the target nucleotide sequence comprises at least a portion of an essential bacterial gene that is needed for survival of the target bacterium.

33. The temperate bacteriophage of claim 32, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK.

34. The temperate bacteriophage of any one of claims 28-29, wherein the target nucleotide sequence is in a non-essential bacterial gene or genomic locus.

35. The temperate bacteriophage of any one of claims 28-34, wherein the first nucleic acid sequence is a first CRISPR array further comprising at least one repeat sequence.

36. The temperate bacteriophage of claim 35, wherein the at least one repeat sequence is operably linked to the first spacer sequence at either its 5′ end or its 3′ end.

37. The temperate bacteriophage of any one of claims 28-36, wherein the first nucleic acid is inserted into a non-essential bacteriophage gene or other genomic locus.

38. The temperate bacteriophage of claim 37, wherein the non-essential bacteriophage gene is gp49, gp75, hoc, gp0.7, gp4.3, gp4.5, gp4.7, gp0.6, gp0.65, gp0.7, gp4.3, or gp4.5.

39. The temperate bacteriophage of any one of claims 28-38, wherein the target bacterium is C. difficile.

40. The temperate bacteriophage of any one of claims 28-39, wherein the temperate bacteriophage is ϕCD146 or ϕCD24-2.

41. The temperate bacteriophage of any one of claims 28-40, wherein the target bacterium is killed by the lytic activity of the temperate bacteriophage, by the activity of a CRISPR-Cas system using the first spacer sequence or the crRNA transcribed therefrom, or both.

42. The temperate bacteriophage of claim 41, wherein the CRISPR-Cas system is endogenous to the target bacterium.

43. The temperate bacteriophage of claim 41, wherein the CRISPR-Cas system is exogenous to the target bacterium.

44. The temperate bacteriophage of any one of claims 41-43, wherein the CRISPR-Cas system is a Type I CRISPR-Cas system, a Type II CRISPR-Cas system, or a Type III CRISPR-Cas system.

45. The temperate bacteriophage of any one of claims 41-44, wherein the CRISPR-Cas system is a Type I CRISPR-Cas system.

46. A pharmaceutical composition comprising:
(a) a bacteriophage of any one of claims 1-27, or a temperate bacteriophage of any one of claims 28-45; and
(b) a pharmaceutically acceptable excipient.

47. The pharmaceutical composition of claim 46, wherein the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, or any combination thereof.

48. A method for killing a target bacterium, the method comprising introducing into the target bacterium a temperate bacteriophage comprising a first nucleic acid sequence encoding a first spacer sequence or a crRNA transcribed therefrom, wherein the first spacer sequence is complementary to a target nucleotide sequence from a target gene in the target bacterium, provided that the bacteriophage is rendered lytic by a 1247 cI repressor region knockout, thereby killing the target bacterium.

49. The method of claim 48, wherein the temperate bacteriophage infects multiple bacterial strains.

50. The method of any one of claims 48-49, wherein the target nucleotide sequence comprises all or a part of a promoter sequence for the target gene.

51. The method of any one of claims 48-49, wherein the target nucleotide sequence comprises all or a part of a nucleotide sequence located on a coding strand of a transcribed region of the target gene.

52. The method of any one of claims 48-49, wherein the target nucleotide sequence comprises at least a portion of an essential bacterial gene that is needed for survival of the target bacterium.

53. The method of claim 52, wherein the essential bacterial gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, or metK.

54. The method of any one of claims 48-49, wherein the target nucleotide sequence is in a non-essential bacterial gene or genomic locus.

55. The method of any one of claims 48-54, wherein the first nucleic acid sequence is a first CRISPR array further comprising at least one repeat sequence.

56. The method of claim 55, wherein the at least one repeat sequence is operably linked to the first spacer sequence at either its 5′ end or its 3′ end.

57. The method of any one of claims 48-56, wherein the first nucleic acid is inserted into a non-essential bacteriophage gene.

58. The method of claim 57, wherein the non-essential bacteriophage gene is gp49, gp75, hoc, gp0.7, gp4.3, gp4.5, gp4.7, gp0.6, gp0.65, gp0.7, gp4.3, or gp4.5.

59. The method of any one of claims 48-58, wherein the target bacterium is C. difficile.

60. The method of any one of claims 48-59, wherein the temperate bacteriophage is ϕCD146 or ϕCD24-2.

61. The method of any one of claims 48-60, wherein the target bacterium is killed by the lytic activity of the temperate bacteriophage, by the activity of a CRISPR-Cas system using the first spacer sequence or the crRNA transcribed therefrom, or both.

62. The method of any one of claims 48-61, wherein the target bacterium is killed by the activity of the CRISPR-Cas system independently of the lytic activity of the temperate bacteriophage.

63. The method of any one of claims 48-62, wherein activity of the CRISPR-Cas system supplements or enhances lytic activity of the temperate bacteriophage.

64. The method of any one of claims 48-63, wherein lytic activity of the temperate bacteriophage and activity of the CRISPR-Cas system are synergistic.

65. The method of any one of claims 48-64, wherein lytic activity of the temperate bacteriophage, activity of the CRISPR-Cas system, or both is modulated by a concentration of the temperate bacteriophage.

66. The method of any one of claims 48-65, wherein the CRISPR-Cas system is endogenous to the target bacterium.

67. The method of any one of claims 48-65, wherein the CRISPR-Cas system is exogenous to the target bacterium.

68. The method of any one of claims 48-67, wherein the CRISPR-Cas system is a Type I CRISPR-Cas system, a Type II CRISPR-Cas system, or a Type III CRISPR-Cas system.

69. The method of any one of claims 48-68, wherein the CRISPR-Cas system is a Type I CRISPR-Cas system.

70. The method of any one of claims 48-69, wherein the temperate bacteriophage does not confer any new properties onto the target bacterium beyond cellular death caused by the lytic activity of the temperate bacteriophage, beyond the activity of the CRISPR-Cas array, or both.

71. A method of treating a disease in an individual in need thereof, the method comprising administering the pharmaceutical composition of any one of claims 46-47.

72. The method of claim 71, wherein the individual is a mammal.

73. The method of any one of claims 71-72, wherein the disease is a bacterial infection.

74. The method of claim 73, wherein a bacterium causing the bacterial infection is an Escherichia coli, Salmonella enterica, Bacillus subtilis, Clostridium acetobutylicum, Clostridium ljungdahlii, Clostridioides difficile, Clostridium bolteae, Acinetobacter baumannii, Mycobacterium tuberculosis, Mycobacterium abscessus, Mycobacterium intracellulare, Mycobacterium fortuitum, Mycobacterium chelonae, Mycobacterium avium, Mycobacterium gordonae, Myxococcus xanthus, Streptococcus pyogenes, cyanobacteria, Staphylococcus aureus, methicillin resistant Staphylococcus aureus, Streptococcus pneumoniae, carbapenem-resistant Enterobacteriaceae, extended spectrum beta-lactamase (ESBL)-producing Enterobacteriaceae, Staphylococcus epidermidis, Staphylococcus salivarius, Corynebacterium minutissimum, Corynebacterium pseudodiphtheriticum, Corynebacterium striatum, Corynebacterium group G1, Corynebacterium group G2, Streptococcus mitis, Streptococcus sanguinis, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces israelii., Porphyromonas gingivalis., Prevotella melaninogenicus, Helicobacter pylori, Helicobacter felis, Enterococcus faecalis, Enterococcus faecium, Enterococcus gallinarum, Bacteroides fragilis, Bacteroides thetaiotaomicron, Fusobacterium nucleatum, Ruminococcus gnavus, or Campylobacter jejuni or any combination thereof.

75. The method of claim 73, wherein the bacterium is a drug resistant bacterium that is resistant to at least one antibiotic.

76. The method of claim 73, wherein the bacterium is a multi-drug resistant bacterium that is resistant to at least one antibiotic.

77. The method of any one of claims 75-76, wherein the at least one antibiotic comprises a cephalosporin, a fluoroquinolone, a carbapenem, a colistin, an aminoglycoside, vancomycin, streptomycin, or methicillin.

78. The method of any one of claims 71-77, wherein the administering is intra-arterial, intravenous, intramuscular, oral, subcutaneous, inhalation, or any combination thereof.


SEQUENCE LISTING (5 sequences)

SEQ ID NO: 1 - DNA, 331 bp, Artificial Sequence
  Description of Artificial Sequence Synthetic polynucleotide
gtgcttttaaatttacaaagtattccattttaattttatagtttagattttatgatataataaaaatatagaagttttgcagtgtgcgatatttgttacaaagtagggcttaatacttgaaatctaagatgttgagggtgcgtgataagtgttatcaattgcactattgcccgctcactgcaattttaagagtattgtatatatgtaggtattggaaatgctaagtttattttggggttttagattaactatatggaatgtaaatggtctagcagctgatattgcatctgctgctgtaactagttttagattaactatatggaatgtaaat

SEQ ID NO: 2 - DNA, 642 bp, Unknown
  Description of Unknown CD24-2 sequence
acattcgaattatttatgtaattttctttttttaatatattcatcagcaaaataatttactaaatatttttcctccaatcctaaagctatttctaattcaataagagcagggatagttaaatctttattctcattattttccaacctagaaatttgacttctgtggcaaccaactctttttgctaactctatttgtgttaatttcttcttttttcgtaactcttttaacatatatttaacctaccttttagataattttatatatgttagtttgtgtaatttcttgtaataaatgtgcaatattcgcacactttttgttggaaatttgtgctagaatgtagttaagaaataactttatctagataaagcaaaaataatagaagctataaaatataataatattttacagttattttatagttttaagctttgaaaaagaggtggttgtaactatttttaagaacgtatgtttcgtggaattaataaataaaatattttataggggatgatatatttgaataagacgaaatattatgatattttaaagttaaacttattaatgaaaaagttgaaagaattggataaaaataaaatcaatgaatataaaataaaagtgaaagaaatacataaaatcaataaaaa

SEQ ID NO: 3 - DNA, 321 bp, Unknown
  Description of Unknown CD24-2 sequence
acattcgaattatttatgtaattttctttttttaatatattcatcagcaaaataatttactaaatatttttcctccaatcctaaagctatttctaattcaataagagcagggatagttaaatctttattctcattattttccaacctagaaatttgacttctgtggcaaccaactctttttgctaactctatttgtgttaatttcttcttttttcgtaactcttttaacatatatttaacctaccttttagataattttatatatgttagtttgtgtaatttcttgtaataaatgtgcaatattcgcacactttttgttgg

SEQ ID NO: 4 - DNA, 3210 bp, Unknown
  Description of Unknown CD24-2 sequence
tgacaaaggttatttggctagggatattataaaatttcaatcttattataaaataataatagataatgaagaatattatgcttttaagaaaaaggtactaaaatctagtaaaagtagaaaaataatgcgttcacttaccactacgcataaaaatgtaagtaaagagaagttagactcattagaagagtactataaaaacttaaaagaatgatatggtaaaaattggatggaattatttaccttaattctcttgtttgtaaaactttattgatatagaatttaacatatacataaatcggtaaaggggtgtagactatgaaaattagtaaattaccagaagcggaattaaaagttatgagatatatttgggaagctaacaaggtattaacttctagggaattagttgctgcaatggaagaaaaatatgaatggaatgaatcaacgacatttacagttttaaaaaggttagagcgaagagaatttttaagcacagagaaaataggcaaacttacacattacaatatactagtaaaagagaaaaaatatctaaggtttgaaacgaaagaatttttgaagaacatacataagaattcaatttcaagtttaatatcagcgttacatagtaaagatgatgaagtggatgaagataaattaattgaactcgaagaacattttaaaaatttaaaagagaaagaataaataaagattattttaggtggcaaattatttttgccaccttttttgccaccatgacagtttaataaaataaaatacaaataaagaaatataaataaaatagaataaagaaattgttttagataggttgtaaataaatgtactcataaaaacataaataaatataaataaacataaataaaatataagaatttctgtgagaaatacgacttttttatataaaatagcaaaataacagtgtttgagcaaaaaaaagaatgttttttatcgttttgccacctgtttgccaccataacttttttcggtggcaaattattgtaaaatgccctccaaaatatctattgtttcactttccattttacttgtaacatgtgaatatgtgtccatagtggttgataattgactatgacctaatctttgttgtatatattttacattagctccattttctaataataatgtggcatgtgtatgtcgcaagcaatgaaaattaaagttaatatttagtttcttgttaatagttcttatagcagcgtcaatattgttgtgatttacaaatgagccatctttttttctacaaacccaatcatactcagtttctttataccattttccaattttaattttttgttttttttgattcaatttttcctcttttaatatcctagacaaagtatcacctattttaatatctcttattgatgtttttgtcttaggtgatgctaattcaaattctgaaacttttctctttattaaattttttctaactttgattatattattatctaaatcaacattatcccaacaaagacctaatatttctccttttctcatgcctgtatgaaaccctataagtagaggaatatagatatttgtatttttaggatatatttctagtattttattgaactcatctagtgttatagtcttgttatctgactcattttttacctttaaaacattttttggtatactggcatattgaacaggattttccttaatgagtttgtaagggtaaacagcagattttaaagcagcatttaatacaacataaatcgcttttaaaactccttttgtgtagtgtttttcttctccgttttgtgtgtatgtctcttttgatttattatttaaaaattcttgaataatagctggatttatagattttagcttacatttaccaagtctaggttctatatgattttcaattaaatttctataactttcctgagtattgtatttgcaattaagaagtacatactctttgtaccaaaagttaagatagtctgataaactcatatttgtttcatcaaacactataccagcattttcatattcatttattgcatcacggagcgatttttcagcttctttcttagtgtttccaccaactctttctaccttttttctcttgccatctactatacctgcatcaaagtagtaataccacttctttccacgttttcttacgccgcctttcataaaatctctcctctcaaaagctttttttatgtaattatataataacatatatcaatttaaataaaataaataaaaaagactataagaaacaaagttttatgtttataattaatagtcttttttatttagatacatatttaatgttttttatcacgtgattccagcactttttctgccaaataaacaactattttttcatactcaatatcagtttctaaagaaaagtcaacaatagcaggtatcgtcatatctcctgcatatttattacctaaccaattttcaattaattccaattcttctttgcttaattcaaattttttctttttattaaaacttaagttcaatttattcacctcaaataatattatatattaaaaatagtatttgatagaaacattcgaattatttatgtaattttctttttttaatatattcatcagcaaaataatttactaaatatttttcctccaatcctaaagctatttctaattcaataagagcagggatagttaaatctttattctcattattttccaacctagaaatttgacttctgtggcaaccaactctttttgctaactctatttgtgttaatttcttcttttttcgtaactcttttaacatatatttaacctaccttttagataattttatatatgttagtttgtgtaatttcttgtaataaatgtgcaatattcgcacactttttgttggaaatttgtgctagaatgtagttaagaaataactttatctagataaagcaaaaataatagaagctataaaatataataatattttacagttattttatagttttaagctttgaaaaagaggtggttgtaactatttttaagaacgtatgtttcgtggaattaataaataaaatattttataggggatgatatatttgaataagacgaaatattatgatattttaaagttaaacttattaatgaaaaagttgaaagaattggataaaaataaaatcaatgaatataaaataaaagtgaaagaaatacataaaatcaataaaaa

SEQ ID NO: 5 - DNA, 4280 bp, Unknown
  Description of Unknown CD24-2 sequence
atatgatataataaaaaagtaaatatgtaaccccaatacatctttactaagtcaaacattattatatagagcattcttcattatggagagtgctttttcatttctttgaataatcatgttgattatttataatattttcatttaatttatctttatctatcaaatttaatgcacaattaatacaaatgttagttttcatatgacttttcttatgaaaagtaatatacttattatcatctttatttattcctttattacagtaatcacataatatagtcttagtcatatttttattcctttcattttatattttccataattaagtttaacatatttagtatatacctatgtatatatatttaaaacaatgttttttattgcaatatagaatatattgtataaataaaaaaatatggagaatggatatttgataattgtaaaattatgttataatttatatgtaaggaactaaatatctaagagtggcagtttccacattatgctctaacctactttctaatgaaaggaggtgggaagtgtggaaatagtagcagtttattacatagcaaaatttttacttgatttatacaaagtaaaaagccactcaaagtcggaagctaagagtggctttatatttaatttaactattaataaactaatagtaaaaattaaatataaaaagtactaaattttttcggaaactacactctcgacaatagattatagttccttatttctaaagaggtaggagaattgcagttctcttatctctttttgatacgaatttacatctttattatatcgcattttgagaaaaaataaaactatgaatattagaaatatattttattttatctctaagtatatatattattagccacctaaaaaactttcacaaaaacaaaaagagtatatgtttttgataactggatgatataacttataactcattttacaattcttagttcatatatatttatgccactaaatttctctagttttgccgatttagttttattctacaactaaaataatacaatagaattagttattattcaacaagattgtttgaaagttaaataaatttatagatatgaacattattttacacttaaaaaaacagactaaatatgtagaatatttggtaaaacttacctctagtttttgtcttaaaaagctcctattattaatttaagttagacgaatttaaggaagtgacataaattgaaagaagaaatattaatagaaaaattattaagagcagaattaatagtaatggagtatttatggaaaaaggacactctaatgcctaaaaaagaaattgtaaaagaaataaaacagatttatggatggcataaaagtacaataaaaatactactaaaaaggctaattgacaaaggttatttggctagggatattataaaatttcaatcttattataaaataataatagataatgaagaatattatgcttttaagaaaaaggtactaaaatctagtaaaagtagaaaaataatgcgttcacttaccactacgcataaaaatgtaagtaaagagaagttagactcattagaagagtactataaaaacttaaaagaatgatatggtaaaaattggatggaattatttaccttaattctcttgtttgtaaaactttattgatatagaatttaacatatacataaatcggtaaaggggtgtagactatgaaaattagtaaattaccagaagcggaattaaaagttatgagatatatttgggaagctaacaaggtattaacttctagggaattagttgctgcaatggaagaaaaatatgaatggaatgaatcaacgacatttacagttttaaaaaggttagagcgaagagaatttttaagcacagagaaaataggcaaacttacacattacaatatactagtaaaagagaaaaaatatctaaggtttgaaacgaaagaatttttgaagaacatacataagaattcaatttcaagtttaatatcagcgttacatagtaaagatgatgaagtggatgaagataaattaattgaactcgaagaacattttaaaaatttaaaagagaaagaataaataaagattattttaggtggcaaattatttttgccaccttttttgccaccatgacagtttaataaaataaaatacaaataaagaaatataaataaaatagaataaagaaattgttttagataggttgtaaataaatgtactcataaaaacataaataaatataaataaacataaataaaatataagaatttctgtgagaaatacgacttttttatataaaatagcaaaataacagtgtttgagcaaaaaaaagaatgttttttatcgttttgccacctgtttgccaccataacttttttcggtggcaaattattgtaaaatgccctccaaaatatctattgtttcactttccattttacttgtaacatgtgaatatgtgtccatagtggttgataattgactatgacctaatctttgttgtatatattttacattagctccattttctaataataatgtggcatgtgtatgtcgcaagcaatgaaaattaaagttaatatttagtttcttgttaatagttcttatagcagcgtcaatattgttgtgatttacaaatgagccatctttttttctacaaacccaatcatactcagtttctttataccattttccaattttaattttttgttttttttgattcaatttttcctcttttaatatcctagacaaagtatcacctattttaatatctcttattgatgtttttgtcttaggtgatgctaattcaaattctgaaacttttctctttattaaattttttctaactttgattatattattatctaaatcaacattatcccaacaaagacctaatatttctccttttctcatgcctgtatgaaaccctataagtagaggaatatagatatttgtatttttaggatatatttctagtattttattgaactcatctagtgttatagtcttgttatctgactcattttttacctttaaaacattttttggtatactggcatattgaacaggattttccttaatgagtttgtaagggtaaacagcagattttaaagcagcatttaatacaacataaatcgcttttaaaactccttttgtgtagtgtttttcttctccgttttgtgtgtatgtctcttttgatttattatttaaaaattcttgaataatagctggatttatagattttagcttacatttaccaagtctaggttctatatgattttcaattaaatttctataactttcctgagtattgtatttgcaattaagaagtacatactctttgtaccaaaagttaagatagtctgataaactcatatttgtttcatcaaacactataccagcattttcatattcatttattgcatcacggagcgatttttcagcttctttcttagtgtttccaccaactctttctaccttttttctcttgccatctactatacctgcatcaaagtagtaataccacttctttccacgttttcttacgccgcctttcataaaatctctcctctcaaaagctttttttatgtaattatataataacatatatcaatttaaataaaataaataaaaaagactataagaaacaaagttttatgtttataattaatagtcttttttatttagatacatatttaatgttttttatcacgtgattccagcactttttctgccaaataaacaactattttttcatactcaatatcagtttctaaagaaaagtcaacaatagcaggtatcgtcatatctcctgcatatttattacctaaccaattttcaattaattccaattcttctttgcttaattcaaattttttctttttattaaaacttaagttcaatttattcacctcaaataatattatatattaaaaatagtatttgatagaaacattcgaattatttatgtaattttctttttttaatatattcatcagcaaaataatttactaaatatttttcctccaatcctaaagctatttctaattcaataagagcagggatagttaaatctttattctcattattttccaacctagaaatttgacttctgtggcaaccaactctttttgctaactctatttgtgttaatttcttcttttttcgtaactcttttaacatatatttaacctaccttttagataattttatatatgttagtttgtgtaatttcttgtaataaatgtgcaatattcgcacactttttgttgg